(19) United States
(12) Patent Application Publication
(10) Pub. No.: US 2004/0082079 A1
(43) Pub. Date: Apr. 29, 2004

(54) Low affinity screening method

(76) Inventors: Ralf Besenbruch, Zweibrucken (DE); Holger Ottleben, Heidelberg (DE); Harald Rau, Heidelberg (DE); Michael Frank, Dossenheim (DE); Renate Sekul, Ladenburg (DE); Dirk Vetter, Heidelberg (DE); Gunther Metz, Heidelberg (DE); Sabine Maier, Heidelberg (DE)

Correspondence Address: Andrew S Marks, 47 Rice Road, Wayland, MA, 01778, US

(21) Appl. No.: 10/467,297

(22) PCT Filed: Feb. 5, 2002
(86) PCT No.: PCT/EP02/01184

(30) Foreign Application Priority Data
Feb. 5, 2001 (EP) 01 10 2522.8

(51) Int. Cl. G01N 33/53; G01N 33/543

(52) U.S. Cl. 436/518; 435/7.1

(57) ABSTRACT

A parallel high throughput screening method on a solid support is disclosed that allows the detection of low affinity binding partners, comprising the steps of:(a) providing a library of different ligands;(b) forming a binding matrix comprising the ligands on a solid support by immobilising said ligands on the support; (c) contacting a target of interest with said binding matrix; (d) parallely determining a binding value of the ligand/target interaction for each type of ligand comprised in the binding matrix; (e) selecting those ligands the binding value of which in an immobilised state towards the target exceeds a predetermined threshold; (f) evaluating the affinity of each of the ligands selected in step (e) in a non-immobilised state towards the target; (g) identifying at least one ligand of step (f) as low affinity binding ligand.

The disclosed invention describes a parallel high throughput screening method on a solid support that allows the detection of low affinity binding partners.

It is generally accepted that the drug discovery process involves the analysis of a multitude of chemical compounds in order to identify a potential drug candidate. For this purpose, biomolecular interactions of the chemical compounds are often studied via target-ligand systems, the target typically being a biomacromolecule (e.g. a protein) and the ligand being a “probe”, i.e. usually a low molecular weight molecule (peptide, oligonucleotide, or so-called small organic molecule). Such ligands exhibit specific structural features which may interact with the target if the latter possesses corresponding structural elements.

In order to analyse the hundreds or thousands of compounds comprising a compound library, screening assays have to be adapted for high-throughput-screening (HTS) which is usually based on microplate systems and robotic liquid handling technology. However, conventional HTS methods can usually be applied only if the target has been validated and functionally characterised. Often, a ligand or substrate has to be known for the target. These HTS methods often allow the detection of high affinity binding molecules, are biased towards screening complex molecules and usually have low hit rates. Even if these technical obstacles have been solved, the analysis of the results is often tedious, as traditional HTS systems often yield false positive results, either because of experimental artefacts or because of interactions of chemical compounds with components of the assay system.

HTS can be performed in solution or on solid phase. The main advantage of solid phase screening is the inherent potential towards miniaturisation of the assay equipment. Another advantage relies on the possibility to reuse the solid support together with the immobilised interaction partner for screening purposes once the structures bound in a first screening run have been removed, e.g. in a washing step. For solid phase screening, either the predominantly macromolecular target or the candidate target binding molecule, i. e. the ligand can be immobilised. While the first alternative is already in use in order to detect high affinity binding partners, the second alternative of immobilising the ligand is considered to be unsuitable for screening because of the putative steric hindrance of the interaction target (see Gordon E. M. and Kerwin J. F., Combinatorial Chemistry and Molecular Diversity in Drug Discovery, Wiley-Liss 1998, p. 424-425).

Another important aspect of HTS screening is the parallelisation of the screening process in order to be able to screen a larger number of compounds per time unit. In this approach, detection systems are used which are capable of recording a plurality of samples simultaneously, for example imaging systems that utilise CCD cameras.

Using such an HTS method, a variety of potential drug candidates included in a compound library can be tested for their capability of interacting with a target. However, the vast palette of available reagents strongly increases the size of theoretically accessible libraries of chemical compounds. As a result of the human genome project more targets are available than can be studied by x-ray crystallography, nuclear magnetic resonance or other high resolution biophysical techniques. It is also very difficult to provide suitable compound libraries for screening methods especially function-blind to investigate this high number of targets. Therefore, a very large number of targets, for which in most cases no functional data are available, need to be studied without suitable information regarding preferred structural features of the candidate ligands.

Any attempt to identify ligands to targets of unknown structures requires libraries of molecules which form a representative subset of the extremely large family of chemical compounds of potential interest. In these subsets, structural diversity should be as high as possible. Diversity criteria can be e.g. atom connectivity, physical properties, computational measurements, or bioactivity as well. The obvious advantage of selecting a subset of compounds that best represents the full range of chemical diversity present in the larger population is to avoid the time and expense of synthesising and screening redundant compounds.

As a consequence, extremely large libraries must be reduced to smaller subsets in order to accommodate current limitations of synthesis and screening facilities which requires a selection of a set of compounds most representative of the entire library. This process of compound selection, called “library design”, can be done randomly, guided by medicinal chemistry or computer-aided.

As outlined above, important criteria for library design have been library size and diversity. More recently, molecular properties related to “drug-likeness” play an increasing role in order to eliminate compounds that have a high chance of failure in the later stages of drug development. Drug-like properties have been widely associated with the so-called ADMET (absorption, distribution, metabolism, excretion, toxicity) rules. These are most commonly defined using the “rule of 5” based on properties of known drugs (Lipinski, C. A et al., Adv. Drug Deliv. Rev. 1997, 23: 3-25).

Recently, a different approach towards library design based on properties of identified leads instead of drugs has been introduced (Teague et al., Angew. Chem. Int. Ed. 1999, 38: 3743-48). A lead compound can be considered as a starting molecule to create analogue compounds for the subsequent identification of a drug. One group of lead compounds are classified by Teague et al. as having low-affinity (>0.1 μm), low molecular weight (<350) and low clogP (negative logarithm of the n-octanol/water partition). Such lead-like compounds are strongly superior to drug-like compounds or even larger substances as often found in classical screening libraries. The reason for this lies in the lead optimisation phase. Detected hits with substantial affinity with respect to their comparably low molecular size (referred to as lead-like compounds or leads) can be subsequently modified to increase their affinity either by combination of identified compounds or by introducing further functionalities via methods of combinatorial and medicinal chemistry to finally yield drug-like compounds with high affinity towards the target. This strategy is in contrast to the situation where a large, complex albeit good binder has to be modified without knowing which of its functionalities are affinity related and, consequentially, have to be retained. However, the implementation of this newly proposed strategy by Teague et al. requires effective methods of screening for low-affinity compounds.

Besides finding suitable methods for library design, the question of how this multitude of compounds (libraries) can best be obtained has to be solved, i.e. synthesis pathways have to be chosen for compounds which are not commercially available.

Combinatorial chemistry offers the best tools for the synthesis of highly diverse libraries. The advantage of combinatorial chemistry, particularly the efficacy of automated parallel synthesis, lies in its ability to produce hundreds and thousands of compounds on a very short time frame. In order to screen the very large numbers of compounds generated by combinatorial chemistry, biological assays have been adapted for HTS.

As compared to a library of drug-like compounds, the members of a library of compounds used in combinatorial chemistry usually exhibit a less complex structure of lower molecular weight. A combinatorial library, i. e. a set of molecules having, e.g., specific chemical functionalities or specific steric structures, typically consists of different building blocks (also referred to as R-groups or monomers, or, in a form where they are not yet connected to the remaining molecule, as reagents). In one approach, these building blocks are combinatorially attached to a common scaffold which, in the case where building blocks are directly connected, may be reduced to the bond created between them upon their reaction. Building block selection can either be based on properties of the building blocks themselves or on the properties of the generated products. While the former method is computationally easier to tackle, working in product space is expected to better cover the essence of a library. Selection of products solely based on their molecular properties (“cherry-picking”) typically results in poor efficiency with respect to a combinatorial plate layout Hybrid methods are available that work in product space but at the same time can be tailored to retain synthetic simplicity, i.e. a limited number of monomers used in synthesis (Pearlman R. S. and Smith K. M., Drugs of the Future 1998, 23: 885-895; Jamois et al., J. Chem. Inf. Comput. Sci. 2000, 40: 63-70).

A distinction between types of combinatorial libraries can be made with regard to size and complexity of the scaffold and the building blocks used. With a large, complex scaffold the blocks can be seen as “decorations” with less intrinsic information where the interplay of scaffold and building blocks predominantly provides the relevant structural features for target affinity. On the other hand, libraries consisting of directly connected building blocks should be constructed from “information-rich”, i. e. relatively complex building blocks that already bear the potential of a certain affinity to the target. They can be considered as ligand fragments representing individual parts of the type of molecule(s) ultimately envisioned as outcome of the screening campaign. In this case, aspects of reagent selection based on the properties of the building blocks themselves gain importance. Of course, these two prototype library concepts are only two extremes with many possible designs in-between.

Concepts and experimental techniques have been introduced for the identification of privileged building blocks such as “SAR by NMR” (Shuker et al., Science 1996, 274: 1531-1534), “SHAPES” (Fejzo et al., Chemistry and Biology 1999, 6: 755-769) and RECAP (Lewell et al., J. Chem. Inf. Comput. Sci. 1998, 38: 511-522). Computer-aided (de novo) drug design methods (Joseph-McCarthy D., Pharmacology & Therapeutics 1999, 84: 179-191; Murcko M. A., Practical Application of Computer-Aided Drug Design, ed. Charifson P. S., p. 305-354) follow a similar rational trying to first discover fragments that can then be combined for more potent compounds. As the fragments used in these approaches are of very low molecular weight, they usually only bind with low affinity to their target and have only been accessible in experimental methods which are relatively time consuming and usually require a great deal of information with regard to the target structure and substantial amounts of test material. Therefore, screening methods are required that facilitate the detection of low affinity binding partners in order to identify low molecular weight ligands or fragments thereof. Such methods could be suitably applied in both the aforementioned synthetic fragment-based approach as well as the “lead-like diversity” method of Teague et al..

A screening method suitable as HTS for the detection of low affinity binding partners should fulfil the following criteria: it should allow screening on a solid support via a parallel detection method. Moreover, the background which may result from unspecific binding between the support and the target or unspecific binding between the ligand structure and the target has to be very low in order to allow the detection of low affinity binding interactions.

Several affinity-based methods have been developed that allow low affinity screening which are not suitable for HTS and do not fulfil the remaining criteria mentioned above. In WO98/48264, WO97/18469 and WO97/18471, nuclear magnetic resonance (NMR) based methods for the design and identification of ligands to target molecules are described. A 15N-labelled target molecule is incubated with a single ligand or a mixture of ligands. The binding site to the protein and the binding constant of the ligand can then be estimated by No spectroscopy. By using this method, ligands that bind with low affinity could be identified. The binding constants for the Stromelysin ligands were very low (17 mM und 0.02 mM) (Hadjuk et al., Science 1997, 278: 497-1534), 499). Cross-inking of this low affinity binding ligands resulted in ligands that bind with high affinity (Shuker et al., 1996, Hadjuk et al., 1997). However, because of the low sensitivity of the NMR methodology, large amounts of 15N-labeled protein are required. In order to use all advantages of the method, a complete structural analysis has to precede the screening. Disadvantageous is that the method can only be applied for relatively small proteins (<40 kDa).

Several groups established methods for the identification of ligands in which a protein is incubated in a compound mixture, followed by an identification of the ligand after suitable purification using mass spectroscopy.

Kaur S. et al. (J. Protein Chem. 1997, 16: 505-11) developed a method which uses size-exclusion chromatography to purify the target-ligand complex after incubation with a ligand mixture. The ligands are then separated by reverse-phase chromatography and identified by MS/MS. A similar method that is suitable for the screening of larger, molecular weight based libraries is described in Lenz G. R. et al. (DDT 2000, 5:145-156).

Both methods are restricted in their application by the fact that the complex has to be very stable in order not to dissociate during size-exclusion chromatography. Alternatively to size-exclusion chromatography capillary electrophoresis can be used as described in WO99/34203. WO00/00823 discloses a technique for the detection of ligands with low affinity which are then used as building blocks for the synthesis of libraries of potential dimeres. Disadvantageous is that very high ligand concentrations have to be used (100-1000 fold excess to the protein), thereby increasing the chance of unspecific binding events (E. M. Gordon at Drug Discovery Technologies).

3D-Pharmaceuticals (www.3dp.com/×13HighThroughput.htm) adapted scanning calorimetry for the use as a screening method. Disadvantageous is the large amount of sample necessary.

In U.S. Pat. No. 5,585,277 and U.S. Pat. No. 5,679,582 screening methods that detect conformation changes that occur upon binding of the ligand are described. Disadvantageous is that for each target protein a new assay has to be developed and that the throughput of 5 000 compounds per week is relatively small.

All these screening methods have the disadvantage in common that they are not suitable for solid phase screening. Thus, they can hardly be performed in parallel and miniaturised.

On the other hand, methods for parallel screening on a solid support that are suitable for high throughput screening have been published. They, however, do not provide the necessary sensitivity to effectively allow the detection of low affinity binding partners.

G. McBeath and S. L. Schreiber (Science 2000, 289: 1760-63; G. McBeath et al., J. Am. Chem. Soc. 1999, 121: 7967-68) use a high precision robot designed to manufacture DNA microarrays to spot droplets of thiol-containing small molecules onto maleimide-derivatised glass slides at high spatial densities (1600 spots per cm²). Each slide can then be probed with a differently tagged protein and binding events are detected by a fluorescence-linked assay.

This microarray has been used to measure about 10,000 binding events involving three different proteins on a single glass slide and in a single experiment. However, with the proposed system only affinity interactions with values for K_{D} in the nanomolar to micromolar range were detected.

P. J. Hergenrother et al. (J. Am. Chem. Soc. 2000, 122: 7849-50) use the same method to immobilise alcohol-containing small molecules on glass slides. This array is capable of detecting known ligands from a compound library containing 80 compounds. Disadvantageous is that the ligands are immobilised via hydroxyl-groups. As the alcohol-containing ligands contain several hydroxyl-groups, the regioselectivity of the reaction is not granted, resulting in different presentations of the ligand.

Moreover, the microarrays described by McBeath et al. and Hergenrother et al. use an aminopropyl-silanised surface that does not allow the formation of an ordered self-assembling monolayer (SAM) (M. Grunze et al., J. Adhesion 1996, 58: 43-67). This favours unspecific binding interactions with the target, thereby increasing the number of false positive hits (Tünnemann R 2000, in “Synthese und spektroskopische Untersuchung Silica-gebundener Peptide und organischer Verbindungen und deren Anwendung in der Sensorik”, Dissertation der Fakultät für Chemie und Pharmazie der Eberhard-Karls-Universität Tübingen, p. 44-46). In addition, the ligand density is very high in both experiments, which gives rise to unspecific binding of targets to ligand clusters, thereby increasing the background. Thus, the detection of low affinity ligands is not possible with these methods because of the high background and because of the high ligand density.

Scharn et al. (J. Comb. Chem. 2000, 2: 361-369) describe a method for parallel synthesis and screening of membrane-bound small organic molecules such as 1,3,5-triazines. A microarray created by this method can contain up to 8 000 samples. Ligand-target interactions are detected via an enzyme-linked assay. Disadvantageous of the microarray described by Scharn et al. is that the cellulose membranes only permit a limited combinatorial chemistry. Furthermore, the cellulose membranes used as support produce relatively high background. Besides, the cellulose matrix forms a hydrogel that contains the ligands not only on the surface but also inside the gel. This often results in a diffusion limitation of the interaction between target and immobilised ligand in the highly hydrated organic matrix. Furthermore, the covalent linking of ligands onto the cellulose matrix takes place randomly, making it impossible to optimise the reaction parameters.

The object of the present invention is to provide a method for the determination of the ability of a chemical compound (referred to as a ligand) which is immobilised on a solid support to bind to a target of interest, even if the affinity of the ligand towards the target is low. The present method is therefore particularly useful for identifying ligands of small molecule size and/or low molecular weight, and it is suitable for high throughput screening. Such a screening method is especially well-suited for investigating poorly characterised targets since functional information on the target is not needed a priori for the identification of low affinity ligands.

Thus, the method of the present invention comprises the steps of

(a) providing a library of different ligands;

(b) forming a binding matrix comprising the ligands on a solid support by immobilising said ligands on the support,

(c) contacting a target of interest with said binding matrix;

(d) parallely determining a binding value of the ligand/ target interaction for each type of ligand comprised in the binding matrix;

(e) selecting those ligands the binding value of which in an immobilised state towards the target exceeds a predetermined threshold;

(f) evaluating the affinity of each of the ligands selected in step (e) in a non-immobilised state towards the target;

(g) identifying at least one ligand of step (f) as low affinity binding ligand.

Of course it should be understood that, starting from a given library with a large number of chemical compounds, there is also the possibility to carry out the present method by immobilising only a part of the compounds at one time in step b) and repeating steps b)-d) until the complete compound library has undergone the screening process.

With the method of the present invention, it was found that contrary to the prejudice of prior art it is possible to detect low affinity binding partners via solid phase screening. The screening method disclosed in the present invention fulfils all criteria of a low affinity detection method: ligand/target interactions are detected via a direct binding assay, and the parallel detection method enables high throughput screening. In contrast to standard solid phase screening methods, where the target is usually immobilised, in the present invention it is the ligands that are immobilised on the solid support. This brings about the advantage that the ligands, once immobilised, can be washed to remove any bound target and can then be reused to screen other targets.

In a first step, a library of different potential low affinity ligands can be selected using criteria for library design known in the art, like diversity, drug- or lead-likeness and in particular the size of the building blocks used. It is preferred to use a library of mainly low molecular weight molecules with a number-average molecular weight of less then 400, preferably <380, more preferably <370 and most preferably <350 g/mol as ligands since they usually qualify as low affinity ligands wherein an individual ligand can have a significantly higher molecular weight, but preferably less than 800, more preferred less than 700 g/mol. However, minimum molecular weights of 40, preferably 50 g/mol, sometimes 60 or 75 g/mol are usually required in order to allow a sufficient interaction. Thus, the present invention differs fundamentally from screening methods known in the art in that compounds can be used for the provision of the library of step a) to be immobilised on the solid support which only have minor affinity towards the target, predominantly due to their low molecular weight or their otherwise low complexity (e.g. with regard to their steric structure).

These ligands are immobilised on a solid support, and in the context of the present invention, the term “binding matrix” generally refers to a surface comprising a plurality of different ligands immobilised on such a support. The necessity of solid phase screening and a parallel detection method make the use of microarrays as solid supports, on which the ligands form a regular pattern, particularly favourable. Identical ligands are usually grouped together, such that the final array comprises a number of fields and each field presents one single type of ligand differing from the ligands presented by the adjacent fields. With the types of ligands in the different fields being known, each type of ligand becomes separately adressable in such an array.

Preferably, the ligands are not immobilised directly onto the support, but via so-called anchor molecules that form a self-assembling monolayer (SAM) on the surface of the support. Such a SAM is very resistant to unspecific target-adsorption which strongly reduces the background. This is critical to allow the detection of low affinity binding ligands.

Possible steric effects that might have a negative influence on the determination of the binding value, such as steric hindrance between bound targets or between targets and ligands as well as spurious signals resulting from unspecific binding between targets and ligand clusters are preferably avoided by using a special surface chemistry. When this strategy is applied, “dilution components”, i.e. structures that do not act as ligands, are preferably present on the support Such dilution components present structures within the binding matrix, which, due to their lack of steric or electronic complexity, cannot be expected to bind to the target of interest. Rather, these components serve exclusively to spatially separate the ligands. Especially in this case, the functional surface presented to the target in HTS is well structured, with a controlled density of ligands helping to avoid agglomerations of ligands and ligand-ligand interactions. Moreover, the ordered structure of the molecules forming this binding matrix strongly reduces background signals arising from unspecific binding between the target and the support or the target and the ligands.

Following their immobilisation, the ligands are brought into contact with a solution or suspension of the target of interest. Suitable targets for which the method of the present invention is particularly useful are macromolecules, in particular biomacromolecules, such as proteins in general, enzymes, etc..

Ligand/target interactions can be detected using, e.g., electrochemical, radiochemical, mass-sensitive or optical methods, such as fluoresence or luminesence measurements. Of course, methods allowing the parallel detection by means of a suitable imaging system, such as a CCD camera, are preferably applied. Particularly preferred are label-free detection methods, e.g. surface plasmon resonance.

After screening the compounds of the combinatorial library with regard to their potential to bind to the target, ligands of interest are selected by defining certain thresholds of the binding value obtained in the screening process. The observable binding value depends on the method of detection whereby the more target molecules bind to one type of ligand, the higher is the binding value for this type of ligand. In the claimed method, hits are preferably selected by ranking the molecules pursuant to their binding values, and the threshold of step (e) of the method according to the invention may be deliberately chosen as to include a certain partition of the screened ligands in the evaluation of the following step (f). A software program (Jarray) that supports this selection process is also presented with the present invention.

The binding value, which represents a relative value for the binding strength between the immobilized ligands of interest and the target allows a first estimate of their mutual affinity. However, binding values are usually only characteristic for the type of detection method chosen. On the other hand, it is time consuming to determine actual equilibration constants for a large amount of compounds irrespective of their activity. Thus, according to the method of the invention, potential low affinity ligands are first selected in step e). In order to render the obtained results comparable and to verify that the screened ligands in their free state give rise to similar results as in their immobilized state, they are then evaluated by affinity determination in step f). In this step, an absolute value for the affinity of the ligand towards the target, such as its dissociation constant K_{D}, its association constant K_{A} or the inhibitory constant of the ligand Kᵢ or its IC₅₀ value, is determined in solution with the ligand in a free, non-immobilized form. Such values, obtained according to conventional methods e.g. from the equilibrium in solution between free ligands and targets on the one hand and ligand—target complexes on the other hand are characteristic indicators for the in vivo effectiveness of a chosen ligand.

The affinity-based evaluation, which is significantly rationalized by the information obtained in the screening step, results in the identification of one or more low affinity binding ligand(s). Suitable low affinity ligands which are identified in step f) above are those with the highest potency to form drugs or structural subunits of drugs to inhibit the concerned target. Usually, those among the low affinity ligands are selected the affinity of which towards the target, seen in the context of the original, non-focused library, is relatively strong. However, interesting low affinity ligands are not necessarily only those with the highest affinities among the screened ligands. By choosing suitable evaluation methods such as “Jarray”, structural subunits within the screened ligands can be identified which strongly contribute to an increase in the overall affinity of the ligand, and ligands carrying such structural subunits are likely to be identified and selected in step f) and thus to be included in a focused library. Finally, other factors such as the cost of their production or the question, whether a specific ligand structure is already known in the field will also have to be considered together with the absolute affinity of a given ligand when suitable low affinity ligands are identified.

The structural information obtained by the analysis of identified hits can be used to design a library of more limited size, the ligands of which are structurally similar to the ligands retrieved in the screening process, a so-called focused library. Of course, the ligands identified in the method of the invention can be used as building blocks or reactants in a further step of combinatorial synthesis, i. e. they can be combined with each other or with ligand structures of different types to form ligands of higher molecular weight and higher functional complexity. Similarly, only substructures of the ligands identified from the original library which have proven to be particularly active can be used as building blocks in the provision of new ligands by combining them with each other or with building blocks of new structures. In this latter case, although the structures subjected to the original screening are varied, the molecular weight of the new ligands based on those originally identified in step (g) is not necessarily increased and may even be slightly reduced.

Thus, the original small molecule ligands can be modified towards higher affinity by introducing additional functionality with potential higher affinity towards the given target. The number of ligands resulting from this combination can again be reduced, selecting the most potent representatives by means of the present screening method, thus proceeding towards the final drug structure. Conventional screening methods or biological assays can be used alone or parallely as soon as the members of the library have reached a certain complexity which makes them accessible to these methods.

Accordingly, the method of the present invention may further comprise steps (a′) to (g′), differing from steps (a) to (g) only in that the initial library used in step (a′) comprises ligands derived from those identified in step (g) as set out above. The affinities determined in step (f′) are at least at the same level and preferably higher than those determined in the preceding step (f).

The method of the present invention allows the identification of low affinity ligands which form, together with the chosen target, a complex with a K_{D} of more than 5 μM. Under normal measurement conditions, values exceeding 10, or even exceeding 50 or 100 μM can be obtained for the ligands selected in the screening step. Thus, it should be understood that the method of the present invention allows the selection of promising ligands from libraries of compounds with a significantly reduced complexity compared to conventional libraries of drug-like compounds. With the sensitivity of the present screening step, suitable structural motivs for the provision of drugs can be identified, e.g., at a very early stage of combinatorial synthesis, where the binding values of the concerned compounds are too low to allow their classification by conventional assay strategies. As a consequence, synthetic efforts as well as the more cost—and time-intensive biological assays can concentrate on ligands or functional subunits which have already been proven effective to a certain degree. Furthermore, affinity data obtained for structures suitable as structural subunits in more complex drug-like molecules can be used as a basis for computational methods used to estimate the activity of such molecules.

In the following, preferred structural requirements and embodiments of the screening setup used for the purpose of the present invention shall be explained.

The support used to immobilize the ligands comprises a substrate that is preferably formed by a metal, most preferably a noble metal (silver, palladium, platinum; especially gold) or a substrate the surface of which is at least partly covered with a layer of such a metal. Particularly preferred are gold surfaces. The material used depends on the detection method. If reflection-optical methods, such as surface plasmon resonance (SPR) are used, the preferred substrates are glass or a light transmitting polymer coated with a thin gold film.

Preferably, the immobilized ligands are arranged in a two-dimensional array formate, i.e. on a microarray comprising discrete fields the spatial location of which can be easily identified and addressed. Each location of the array carries one type of ligand from a known source and with a known structure. Suitable microarrays for the purpose of the present invention include, e.g., a two-dimensional planar solid support with a plurality of position-addressable reaction areas for the immobilisation of samples of small size, preferably in a regular pattern, of about less than 2.5 mm, preferably less than 1 mm, more preferably 0.5 mm in diameter, for screening purposes. As regards the number of reaction areas, conventional microplates can be used, such as those of the 96-well or 384-well type. However, in terms of an acceleration of the screening process, the number of reaction areas preferably reaches at least 1536, more preferably at least 3072 or at least 4608 and particularly preferred are 9216.

If a microarray is used as a solid support, the number of different compounds in the initial library of candidate target binding molecules preferably corresponds to the number of reaction areas in the array. For the method of the present invention, libraries comprising at least about 1536, particularly at least 3072 or at least 4608, more particularly at least 9216 different compounds are preferred.

In order to arrive at the binding matrix of the present invention, several approaches are possible. Depending on the structure of the ligands, and on the functional groups they provide, the ligands may be applied directly onto the solid support, thus providing a binding matrix. However, for the purpose of the present invention, the ligands are preferably immobilised on the support via anchor molecules comprising at least two functional moieties at opposite ends of the anchor, one being able to bind with the surface of the support, the other one to bind the ligand. Such anchors should be able to form a self-assembling monolayer (SAM) on the surface of the support. Suitable anchor structures are, e.g., disclosed in WO 00/73796 and DE 100 27 397.1, and those are preferred for the purpose of the present invention which carry a thiol functionality to interact with the solid support. Suitable structural elements that support SAM formation and, at the same time, allow the adjustment of suitable distances between the support and the ligand, are described in DE 199 24 606.8 or WO 00/73796. The above documents also provide a detailed description of methods for the synthesis of such anchors and of suitable binding matrices containing them together with ligands attached to them.

The ligand may be bound, preferably covalently, with the anchor structure prior to its immobilisation on the support. In this case, complete ligand-anchor-conjugates (LAC) are contacted with and bound to the support as disclosed in WO 00/73796.

However, for the present method, the strategy disclosed in DE 100 27 397.1, where the anchor molecules are immobilised on the support in an activated form and are subsequently bound with the ligand, has proven to be particularly advantageous. In this latter approach, anchor structures are synthesized so as to carry a reactive “head group”, i.e. a group which allows a selective and preferably quantitative reaction of the thus activated anchor with the ligand. It should be understood that this head group is at a terminal of the anchor structure facing away from the support on which the anchor is immobilised. Depending on the chemical nature of the head group, this strategy may require a chemical modification of the ligand so as to carry a specific functionality which is able to react with the head group of the activated anchor. Once the activated anchors are immobilised on the solid support, they can be reacted with the ligand/modified ligand in a separate step to provide the binding matrix. Usually this reaction is conducted with an excess of the ligand/modified ligand to get a preferably quantitative conversion of the reactive “head groups” of the anchor molecules. An advantage of this method is that the ligand concentration on the surface is solely determined by the concentration of anchor molecules and not by the concentration of ligands in the added solution. This is of particular advantage if many ligands that are e.g. obtained by combinatorial synthesis and that are present in imprecise concentration have to be analysed in parallel. Therefore, the reproducibility and the comparability of different measurements can be improved. Mercaptophilic head groups as listed in DE 100 27 397.1 which covalently bind the ligand are preferred for this purpose. Among them, the method of providing a binding matrix by reacting a thiol-containing ligand with immobilised anchors carrying a maleimide as a head group has been proven particularly advantageous. In this case, a thiol functionality is introduced into the ligands to be screened during or after their synthesis. Once the surface of the support is covered with the anchor structures, the thiol—functionalised ligands are reacted with the mercaptophilic head group to provide ligand anchor conjugates immobilised on the support.

Thus, anchor molecules of the present invention preferably have the following general structure

HS—R-M (1)

for the definition and synthesis of which, including particularly preferred embodiments, it is referred to DE 100 27 397.1 according to which R is a linear or branched, optionally substituted, saturated or unsaturated hydrocarbon chain which may comprise heteroatoms, aromatics and heterocyclic compounds. It comprises 5-2000 atoms, including heteroatoms. In a preferred embodiment, R in formula (1) comprises one or both of the structural subunits R^{a} and R^{b}, with R^{a} being positioned adjacent to the thiol functionality.

R^{a} is a bivalent moiety, which preferably allows the formation of a SAM and for this purpose it should be largely hydrophobic. It comprises a branched or linear hydrocarbon chain of 5 to 50 carbon atoms which may be completely saturated or partly unsaturated and which may be interrupted by aromatics, heterocyclic compounds or heteroatoms, a completely saturated hydrocarbon chain without heteroatoms being preferred. In a preferred form, it has the general formula —(CH₂)ₙ—, wherein n is an integer from 5 to 50, preferably from 5 to 25, particularly preferably from 5 to 18 and most preferably from 8 to 12.

R^{b}, which is equally bivalent, represents in a first preferred embodiment an oligoether of the general formula —(OAlk)_{y}-, wherein y is an integer and Alk is an alkylene group. A structure wherein y ranges between 1 and 100, preferably between 1 and 20, and most preferably between 2 and 10, is preferred. The Alk group preferably exhibits 1-20, more preferably 2-10 and particularly preferably 2-5 carbon atoms. —(OC₂H₄)_{y}— is most preferred.

In a second preferred embodiment, R^{b} is an oligoamide which is formed by dicarboxylic acids and diamines and/or amino carboxylic acids, wherein the amines independently of each other exhibit from 1 to 20, particularly preferably from 1 to 10 carbon atoms and may also be interrupted by further heteroatoms, in particular oxygen atoms. The carboxylic acid monomers, independently of each other, preferably have from 1 to 20, more preferably from 1 to 10 carbon atoms and may also be interrupted by further heteroatoms, in particular oxygen atoms.

Further preferred are anchor structures wherein either R^{a} alone, or R^{a} and R^{b} together, link HS and M in the above formula (1).

Particularly preferred are groups R of the general formula:

—(CH₂)ₐ-Q¹-(CH₂)_{b}-{(Q²-(CH₂)_{c}—[O—(CH₂)_{d}]ₑ—

O—(CH₂)_{f}]_{g}-[Q³-(CH₂)_{c′}—[O—(CH₂)_{d′}]_{e′}—O—(CH₂)_{f′}]ₕ}ᵢQ⁴-(CH₂)ⱼ-Q⁵-(CH₂)ₖ— (2)

wherein the variables, independently of each other, are defined as follows and numerical ranges are to comprise their respective limiting values as well as all integers in-between:

| | |
|---|---|
| Q¹, Q⁵ | represent —NH—C(O)—, —C(O)—NH— or a bond; |
| Q², Q³, Q⁴ | represent —NH—C(O)— or —C(O)—NH—; |
| a | is from 5 to 20, preferably 8 to 12, particularly preferably 10; |
| b | is from 0 to 5, preferably 0 if Q¹ is a bond and from 1 to 10, preferably 2 to 7, |
| | particularly preferably 3 to 5 in all other cases |
| c, c′ | are from 1 to 5, preferably 1 to 3, particularly preferably 1; |
| d, d′ | are from 1 to 5, preferably 1 to 3, particularly preferably 2; |
| e, e′ | are from 1 to 5, preferably 1 to 3, particularly preferably 2; |
| f, f′ | are from 1 to 5, preferably 1 to 3, particularly preferably 1; |
| g, h | are from 0 to 3, provided that g + h ≧ 1, preferably g + h = 2; |
| i | is from 1 to 3, preferably 1 to 2, particularly preferably 1; |
| j | is from 0 to 5, preferably 1 to 3, particularly preferably 2; and |
| k | is from 0 to 5. |

Mercaptophilic head groups M are, e.g., iodine and bromine acetamides, pyridyldithio compounds, Michael acceptors in general, acrylic acid derivatives such as the esters, amides, lactones or lactames thereof, methylene-gem-difluorocyclopropanes, α,β-unsaturated aldehydes and ketones as well as α,β-unsaturated sulfones and sulfonamides.

Preferred head groups M are those of the general formula

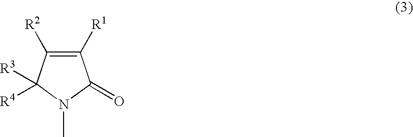

wherein

R¹ and R², independently of each other, represent hydrogen or C₁-C₅ alkyl, preferably methyl, ethyl or n-propyl,

R³ and R⁴, independently of each other, represent hydrogen or C₁-C₅ alkyl, preferably methyl, ethyl or n-propyl, or R³ and R⁴ together are ═O and

the binding to the other anchor is effected via the nitrogen atom.

Preferably, R³ and R⁴ together are ═O, most preferably the head group is a maleimidyl group.

In the final binding matrix, dilution components are preferably present on the surface together with the immobilised ligands in order to control the distance between adjacent ligands. These dilution components do not present ligands or activated groups to allow their immobilisation. Rather, they contribute chemically simple structures to the binding matrix which are unlikely to show any interaction with the target. The dilution of ligands avoids their mutual interaction, which could influence the interaction with the target. At the same time, interaction of bound targets is also avoided due to the spatial separation of the ligands as coupling sites. However, the dilution components should not affect the interaction of the immobilised ligand with the target. Particularly, no binding of the dilution component to the target should occur. Therefore, the dilution component should have a high adsorption resistance towards the target, e.g. a protein. Thus, suitable dilution components have sterically and electronically simple structures, e.g. based on hydrocarbon chains provided with a simple functional group to allow their immobilization on the support.

Particularly suitable functionalized surfaces for solid phase screenig are obtained if the dilution components and the ligands or ligand carrying structures are used in a ratio ranging from 1:2 to 1:10000, preferably from 1:10 to 1:1000 or 1:10 to 1:100. Homogeneously functionalised surfaces are best provided by bringing a well mixed solution of both ligands and dilution components in contact with the support.

In cases where anchor structures are used to immobilize the ligands, the total length of the dilution component should be slightly shorter than that of the anchor molecule. Otherwise, the anchor molecule and the dilution component should have a large structural similarity in order to ensure homogeneous blending on the solid phase surface and to allow the formation of well structured SAMs. Exemplary dilution components that fulfil these criteria have the general formula

HS—R—X (4)

the variables of which are equally defined in DE 100 27 397.1, and they are preferably used for the purpose of the present invention. Thus, while R is independently defined as for the anchor structure above, X is a non-mercaptophilic head group, preferably derived from a small molecule with a molecular weight of less than 60, 50 or even 40 g/mol. Often, C₁-C₄ alkoxy or acylamide groups are used and methoxy groups as well as acetamide groups are particularly preferred. Here, the dilution components and the anchor molecules are preferably used in a ratio ranging from 1:2 to 1:10000, and more preferably from 1:10 to 1:1000 and particularly preferable from 1:10 to 1:100. Again, homogeneously functionalised surfaces are best provided by bringing a well mixed solution of both anchors and dilution components in contact with the support, and it is referred to DE 100 27 397.1. with regard to specific techniques. After this step, the ligands can be bound to the anchor structures. Alternatively, such preferred dilution components can also be used in cases where complete ligand anchor conjugates as described e.g. in WO 00/73796 are used to form the binding matrix. Here, mixed solutions comprising the dilution components together with ligand anchor conjugates are contacted with the support.

Suitable anchor structures to be further modified to carry ligands, ligand-anchor-conjugates and dilution components are preferably provided by solid phase synthesis, followed by cleaving the anchor or a complete ligand-anchor-conjugate from the solid substrate used during its synthesis and contacting it with the solid support used in HTS.

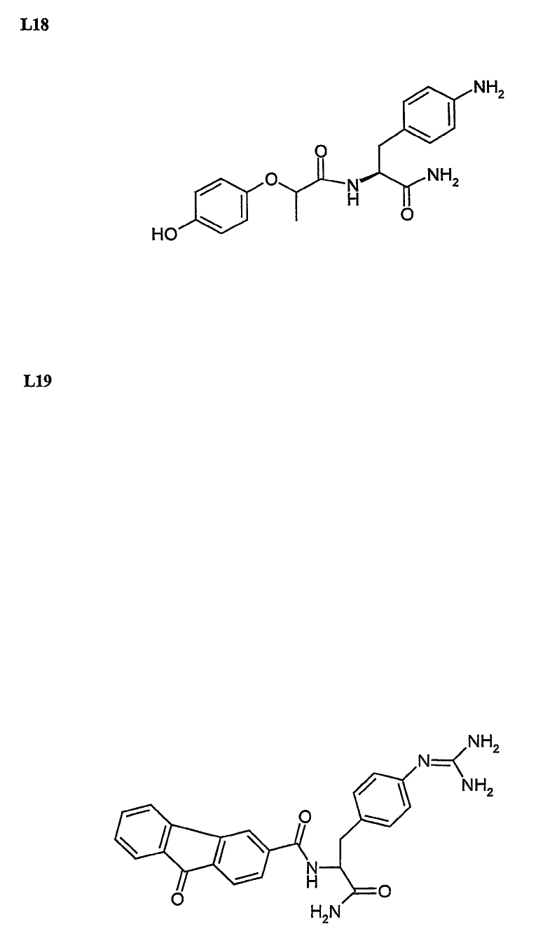

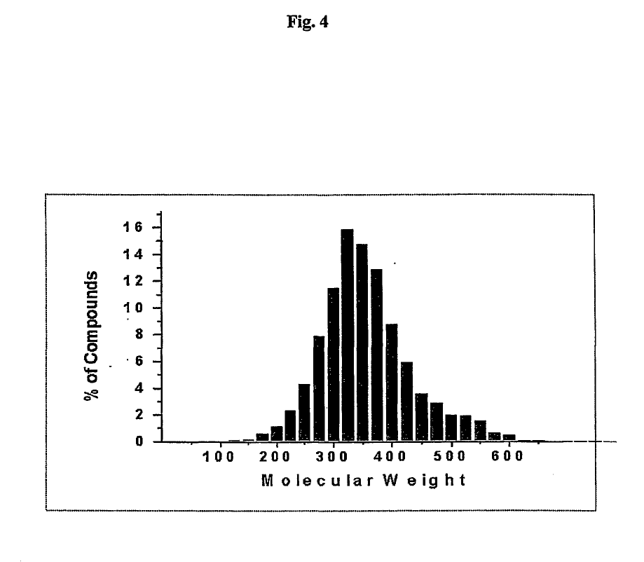

Preferred libraries of ligands to be screened in the method of the invention, as referred to in step (a) above, are designed towards ligands that fall to a large extend into the lead-like classification coined by Teague et al. The number-average molecular weight of the molecules in such initial libraries should be less than 400, preferably <380, more preferably <370 and most preferably <350 g/mol wherein an individual ligand can have a significantly higher molecular weight, preferable less then 800, more preferred less then 700 g/mol. The number-average molecular weight of the ligands in a library is the sum of the weights of the ligands divided by the number of ligands. One example for the molecular weight distribution of such a preferred library is shown in FIG. 4. Also preferred are libraries wherein the ligands share a small common core size. In particular, ligands obtained by forming binary combinations from two sets of reactants, which are directly connected as building blocks e.g. in a step of combinatorial synthesis are preferred. The building blocks formed by the reactants then have average molecular weights ranging from 50 or 75 to 250, preferably from 100 to 150 or 200, particularly preferred from 150 to 200 g/mol.

In the context of the present invention, the term “building block” is intended to refer to substructures of ligands which are introduced into the overall ligand structure in a single reaction, preferably in a single step of combinatorial synthesis. The term “reactant” as used in the context of the formation of ligands, refers to molecules which are not yet incorporated into the ligand and which are used to provide the building blocks.

Besides the molecular criteria, availability of building blocks as well as synthetic feasibility and efficiency are aspects to be considered in designing libraries for screening. Being able to screen for small molecules with an affinity at the micromolar level also facilitates building block selection since the diversity space (i.e. the number of molecules available by variation of the basic functional subunits) is smaller.

Computational methods for library design (Pearlman and Smith, 1998; Jamois et al. 2000) help to cope with the large number of potential compounds which can be synthesised by combinatorial chemistry and which exceeds screening capacities even in the context of ultra high-throughput technologies. In order to describe the complex properties of compound collections such as molecular diversity or structural bias towards a pharmacophoric motif, several molecular encoding schemes have been developed that can be used for computerised storage and processing. Molecular descriptors range from simple whole molecule properties (Molweight, clogP, polarisability) to 2D descriptors representing atom connectivity's (structural keys, fingerprints) and to methods for capturing 3D information (pharmacophore fingerprints). Conceptually, molecules are distributed in a high-dimensional so-called diversity space which is defined by a set of descriptors. Common mathematical methods for compound selection are either based on intermolecular distance together with clustering algorithms. Alternatively, cell-based partitioning methods with prior reduction of the dimensionality are applied (Gorse D. and Lahana R., Current Opinion in Chemical Biology 2000, 4: 287-294; Van Drie J. H. and Lajiness M. S., Drug Discover Today 1998, 3: 274-283).

Preferred ligands to be used in the context of the present invention comprise a structure of the following general formula:

L¹-L², (5)

wherein L¹ and L² represent the building blocks referred to above and are independently formed by an amine, alcohol, carboxylic acid or an amino acid, chosen such that the reactants yielding L¹ and L² have supplementary chemical functionalities which allow the direct formation of a chemical bond. For the method of the present invention, preferably the ligands are not formed from two natural occurring amino acids connected by the condensation reaction of the alpha amino group of one amino acid with the alpha carboxyl group of the second amino acid in the same ligand. Ligands based on those dipeptides can be sensitive to enzymatic degradation during the screening method of the present invention. Drugs developed on the base of those dipeptides are expected to be sensitive to enzymatic degradation resulting in short in vivo half live times.

The ligand is synthesised from two reactants Lᵣ¹ and Lᵣ² (preferably belonging to two different reactant libraries) which yield the corresponding building blocks L¹ and L², respectively. They contain at least one functional group suitable for the synthesis of the desired combinatorial library of ligands and Lᵣ² contains at least one additional functional group suitable to immobilise the ligand on the solid support surface either directly or indirectly via an anchor molecule. The functional groups of Lᵣ¹ and Lᵣ² required for their combination can be independently an amine, an alcohol, a thiol, a carboxylic or a sulfonic group, chosen such that Lᵣ¹ and Lᵣ² have supplementary chemical functionality which allow the direct formation of a chemical bond. Non-limiting examples for supplementary chemical functionalities are the combinations of a carboxylic group and an amine, a carboxylic group and an alcohol, a sulfonyl acid and an amine. It is well known in the art to use such functional groups directly or in activated form (e.g. an acid halide, an anhydride, the reaction product of the carboxylic acid with a carbodiimide or an ester with N-hydroxysuccinimide instead of the carboxylic acid group). Moreover, the reactants Lᵣ¹ and Lᵣ² may comprise protective groups in order to avoid reactions of further functional groups which are to serve for the immobilisation of the ligand or potential interaction with the target During synthesis or at the end of the synthesis of the ligand, the protective groups can be removed. Protective groups for organic chemical synthesis are known by one with ordinary skills in the art including the reagents and conditions for their introduction and for their removal.

The functional group of L² required for the immobilisation of the ligand on the solid support surface can be an amino, a hydroxyl or a thiol group, a carboxylic acid or a sulfonic acid residue. In case anchor molecules are used to bind the ligands in a preferably covalent form, any other functionality of a chemical component capable of forming a covalent bound to corresponding supplementary functionality can additionally be used.

Beside the required functional groups for the synthesis of the ligand and the immobilisation of the ligand to the solid support surface, the reactants Lᵣ¹ and Lᵣ² can contain additional functional groups which may be introduced in a protected form to avoid side reactions during the synthesis of the ligand. Such functional groups represent potential sites for the interaction with the target. Non-exclusive examples for functional groups are —OH, —SH, —S—C1-4-alkyl, —Cl, —F, —Br, CF3, —CN, —CHO, COOH, —COO-C1-4-alkyl, —C1-4-alkyl, —C1-4-alkyloxy, —NO2, —NH2, —NH—C1-4-alkyl, —CONH2, —COHN—C1-4-alkyl, —CON—(C1-4-alkyl)2, —NHCO—C1-4-alkyl, aryl, heteroaryl.

The inventive screening method preferably uses libraries of Lᵣ¹ and Lᵣ² so that the resulting library of ligands comprising the structure L¹-L² fulfils the criteria for the leadlike lead approach of Teague et al. using to a large extend small molecules having a number-average molecular weight Mn of less than 400, preferably <380, more preferably <370 and most preferably <350 g/mol.

Although the inventive screening method can be generally used with a wide range of different targets, the screening method is preferably used for the screening of enzymes and particularly useful for the screening of proteases. Proteases catalyse the cleavage of peptide bonds. Ligands synthesised from an amino acid and a carboxylic acid or sulphonic acid own certain molecular elements common to naturally occurring peptides. Thus, it can be expected that they are able to bind specifically to the active site of proteases and that they are not cleavable at all or not with the same reaction rate by proteases as are natural occurring peptides. Ligands suitable for the inventive screening method should not be cleavable during the screening process by the target to avoid misleading results. Thus in a more preferred embodiment, the Lᵣ¹ is a reactant containing a carboxylic acid group or a sulfonic acid group function. Lᵣ² is an amino acid or amino acid with protective groups where appropriate and the immobilisation is accomplished by a carboxylic functionality of the amino acid. By varying the reactant Lᵣ¹ (“cap”) of these so-called “capped amino acids” the diversity space of the combinatorial library can be increased.

Preferred and non-limiting examples for reactants Lᵣ² are: Fmoc-L-alanine, Fmoc-L-leucine, Fmoc-L-methionine, Fmoc-L-asparagine(Trt) Fmoc-L-proline, Fmoc-L-glutamine(Trt), Fmoc-L-arginine(Pbf), Fmoc-L-serine(tBu), Fmoc-L-threonine(tBu), Fmoc-L-valine, Fmoc-L-tryptophan(Boc), Fmoc-L-cysteine(Trt), Fmoc-D-phenylalanine, Fmoc-L-aspartic acid(OtBu), Fmoc-D-proline, Fmoc-D-glutamine(Trt), Fmoc-L-glutamic acid(OtBu), Fmoc-L-methionine(O2), Fmoc-beta-alanine, Fmoc-L-phenylglycine, Fmoc-D-phenylglycine, Fmoc-L-lysine(Dde), Fmoc-L-cyclohexylalanine, Fmoc-L-phenylalanine, Fmoc-L-methionine-sulfoxide, Fmoc-L-citrulline, Fmoc-L-phosphotyrosine, Fmoc-L-glycine, Fmoc-L-benzoylphenylalanine, Fmoc-L-diaminopropionic acid(ivDde), Fmoc-L-tetrahydroisoquinolinecarboxylic acid, Fmoc-L-2-farylalanine, Fmoc-L-histidine(Trt), Fmoc-L-3-thienylalanine, Fmoc-L-4-thiazolylalanine, Fmoc-L-arginine(NO2), Fmoc-L-isoleucine, Fmoc-isonipecotic acid, Fmoc-L-cyclohexylglycine,. Fmoc-L-lysine(Boc)-OH, Fmoc-L-1-naphthylalanine, Fmoc-L-3-benzothienylanine, Fmoc-D-1,2,3,4-tetrahydronorharman-3-carboxylic acid, Fmoc-4-(aminomethyl)benzoic acid, Fmoc-L-tyrosine(but), Fmoc-L-arginine(Tos), Fmoc-L-hydroxyproline(but), Fmoc-L-ornithine(Boc), Fmoc-L-indoline-2-carboxylic acid, Fmoc-D-alanine, Fmoc-L-glutamic acid(OBzl), Fmoc-L-lysine(Z), Fmoc-L-serine(Bzl), Fmoc-D-glutamic acid(OtBu), Fmoc-D-methionine, Fmoc-D-tyrosinebut), Fmoc-D-tryptophan(Boc), Fmoc-D-histidine(Trt), Fmoc-L-glutamic acid-OtBu, Fmoc-L-glutamic acid gamma-cyclohexyl ester, Fmoc-D-leucine, Fmoc-L-tyrosine(2,6-Cl₂-Bzl), Fmoc-D-arginine(Pbf), Fnoc-L-tyrosine(Bzl), Fmoc-D-cyclohexylalanine, Fmoc-L-aspartic acid(OCHx), Fmoc-L-threonine(Bzl), Fmoc-L-pipecolic acid, Fmoc-D-4-thiazolylalanine, Fmoc-L-diaminobutyric acid(Boc), Fmoc-D-lysine(Boc), Fmoc-L-cysteine(Acm), Fmoc-L-hydroxyproline(Bzl), Fmoc-D-arginine(Mts), Fmoc-L-tyrosine(2-Br-Z), Fmoc-L-tyrosine(3-i), Fmoc-D-1,2,3,4-tetrahydroisoquinoline-3-carboxylic acid, Fmoc-N-methyl-L-serine(Bzl), Fmoc-delta-phenylalanine, Fmoc-D-4-benzoylphenylalanine, Fmoc-3-(1-naphthyl)-D-alanine, Fmoc-L-phenylalanine(4-guanidino-Boc2), Fmoc-D-tyrosine(B3zl), Fmoc-L-cysteine(but), Fmoc-L-cysteine(4-methyl-Bzl), Fmoc-3-(2-naphthyl)-L-alanine, Fmoc-D-thiazolidine-4-carboxylic acid, Fmoc-D-asparagine(Trt), Fmoc-L-phenylalanine(3,4-Cl₂), Fmoc-D-cysteine(Acm), Fmoc-L-tyrosine(3,5-I₂), Fmoc-D-pipecolic acid, Fmoc-L-phenylalanine(4-NH-Boc), Boc-D-phenylalanine(4-NH-Fmoc), Fmoc-L-octahydroindole-2-carboxylic acid, Fmoc-L-2,3-diaminopropionic acid(Boc), Fmoc-L-tyrosine(3-NO2), Fmoc-N-methylglycine, Fmoc-L-histidine(Tos), N-alpha-Fmoc-N-beta-Alloc-L-2,3-diaminopropionic acid, 4-Fmoc-piperazin-1-ylacetic acid hydrate, Fmoc-L-glutamic acid(OMe), Fmoc-L-tyrosine(Me), Fmoc-L-tyrosine(All), Fmoc-L-glutamic acid(OAll), Fmoc-L-tyrosine(3,5-Br2), Fmoc-D-ornithine(Boc), Fmoc-(3-aminomethyl)-benzoic acid, N-Fmoc-2-aminoindane-2-carboxylic acid, Fmoc-L-2-pyridylalanine, Fmoc-L-lysine(ivDde), Fmoc-D-4-iodophenylalanine, Fmoc-cis-2-amino-4-cyclohexene-1-carboxylic acid (racemate), Fmoc-cis-2-amino-1-cyclopentanecarboxylic acid (racemate), Fmoc-(±)-baclofen, Fmoc-1-aminocyclopentane-1-carboxylic acid, Fmoc-6,7-dimethoxy-1,2,3,4-tetrahydro-1-isoquinolineacetic acid (racemate), Fmoc-nipecotic acid (racemate), Fmoc-cis-2-aminocyclohexane-carboxylic acid (racemate), Fmoc-N-(Boc4piperidyl)-glycine, (2S,4S)-Boc4-amino-1-Fmoc-pyrrolidine-2-carboxylic acid, (R,S)-N-Fmoc-N′-Boc-imidazolidine-2-carboxylic acid, Fmoc-L-Cystein(Boc-3-aminopropyl)OH.

Preferred and non-limiting examples for reactants Lᵣ¹ are: mono-methyl cis-5-norbornene-endo-2,3-dicarboxylate (racemate), 4-(1,1,-dioxo-1lambda-6-,4-thiazinan-4-yl)benzene-carboxylic acid, 5,7-dimethylpyrazolo[5,4-a]pyrimidine-3-carboxylic acid, (−)-cis-isoketopinic acid, (−)-menthoxyacetic acid, (±)-pinolic acid, (1,2-dihydro-1-oxophthalazin-4-yl)acetic acid, (1h-benzotriazol-1-yl)acetic acid, (1R)-(+)-camphanic acid, (2,4-dioxo-1,3-thiazolidin-3-yl)acetic acid, (2-benzothiazol-2-ylsulfanyl)acetic acid, (2-methyl-1H-benzimidazol-1-yl)acetic acid, (2-methyl-1-oxo-1,2-dihydro-isoquinolin-4-yl)-acetic acid, (3-ethyl-4-oxo-3,4-dihydro-phthalazin-1-yl)-acetic acid, (3-methoxyphenoxy)-acetic acid, (3S)2-(2-methoxyphenyl)-5-oxotetrahydrofuran-3-carboxylic acid, (4-chlorophenylthio)acetic acid, (4H-1,2,4-triazol-3-ylsulfanyl)-acetic acid, (5-methyl-2-phenyloxazol-4-yl)acetic acid, (E)2-phenyl-3-(2-thienyl)-2-acrylic acid, (E)-5-(2-carboxyvinyl)-2,4-dimethoxy-pyrimidine, (quinolin-2-ylsulfanyl)acetic acid, (R)-(−)-alpha-[[4-ethyl-2,3-dioxo-1-piperazi(nyl)carbonyl]amino]-4-hydroxybenzeneacetic acid, (R)-(+)-citronellic acid, (S)-(+)-O-acetylmandelic acid, [(1-cyclohexyl-1H-tetrazol-5-yl)sulfanyl]acetic acid, [(4-methylquinoline-2-yl)sulfanyl]acetic acid, 1-(2,4-dichlorophenyl)cyclopropanecarboxylic acid, 1-(2-carboxyethyl)-3-methylbenzimidazole-2-(1H)-thione, 1-(2-chlorobenzyl)-5- oxopyrrolidine-3-carboxylic acid (racemate), 1-(2-chlorobenzyl)-6-oxo-1,6-dihydro-3-pyridinecarboxylic acid, 1-(2-pyrimidinyl)-4-piperidinecarboxylic acid, 1-(3,5-dichlorophenyl)-5-methyl-1H-1,2,4-triazole-3-carboxylic acid, 1-(3-carboxypropionyl)indoline, 1-(4-chlorophenyl)-1-cyclopentanecarboxylic acid, 1-(4-methoxyphenyl)ethyliminoxyacetic acid, 1-(6-chloro-3-pyridazinyl)-4-piperidinecarboxylic acid, 1-(carboxymethyl)benzimidazole, 1,2,3-trimethyl-1H-indole-5-carboxylic acid, 1,2-dihydro-3-methyl-2-oxo-4-quinolinecarboxylic acid, 1,4-benzodioxan-2-carboxylic acid (racemate), 1,5-dimethyl-1H-pyrazole-3-carboxylic acid, 1,6-naphthyridine-2-carboxylic acid, 1-acetylpiperidine-4-carboxylic acid, 1-adamantaneacetic acid, 1-cycloundecene-1-carboxylic acid, 1-methyl 2-aminoterephthalate, 1-methyl-3-(trifluoromethyl)-1H-pyrazole-4-carboxylic acid, 1-methyl-3-(trifluoromethyl)-1H-thieno[2,3-c]pyrazole-5-carboxylic acid, 1-naphthalenesulfonyl chloride, 1-phenyl-1-cyclopentanecarboxylic acid, 1-phenyl-5-N-propylpyrazole-4-carboxylic acid, 2-(1,1-dioxo-1lambda˜6˜,4-thiazinan-4-yl)benzene-carboxylic acid, 2-(1-naphthoxy)propionic acid, 2-(2-aminothiazole-4-yl)-2-methoxyiminoacetic acid, 2-(2-chloroacetamido)-4-thiazoleacetic acid, 2(2-furyl)-4-quinolinecarboxylic acid, 2-(2-nitrobenzylthio)acetic acid, 2-(2-phenyl-1,3-thiazol-5-yl)acetic acid, 2-(2-thienyl)-1,3-thiazole-4-carboxylic acid, 2-(4-(tert-butyl)phenoxy)nicotinic acid, 2-(4,6-dimethylpyrimidin-2-ylthio)acetic acid, 2-(4-chlorophenoxy)nicotinic acid, 2-(4-chlorophenyl)-1,3-thiazole-4-carboxylic acid, 2-(4-chlorophenyl)-4-quinolinecarboxylic acid, 2-(4-cyanophenoxy)-2-methylpropionic acid, 2-(-4-fluorophenoxy)pyridine-3-carboxylic acid, 2-(4-hydroxyphenoxy)propionic acid (racemate), 2-(4-methylphenoxy)nicotinic acid, 2-(4-tert-butylphenoxy)acetic acid, 2- (benzylsulfanyl)benzenecarboxylic acid, 2-(methylsulfinyl)benzenecarboxylic acid, 2-(o-chlorophenoxy)-2-methyl-propionic acid, 2-(phenethylthio)acetic acid, 2-(phenylthio)nicotinic acid, 2-(trifluoromethyl)phenylacetic acid, 2,2,5,7-tetrarmethylindan-1-one-4-carboxylic acid, 2,2-dichloro-1-methyl-cyclopropanecarboxylic acid (racemate), 2,2-diphenylpropionic acid, 2,3,4,5,6-pentafluorophenylacetic acid, 2,3-dichloro-4-(ethylsulfonyl)benzenecarboxylic acid, 2,3-dihydro-1H-cyclopenta[b]quinoline-9-carboxylic acid, 2,3-dihydro-3-oxopyridazine-6-carboxylic acid, 2,3-dihydrobenzo[b]furan-5-carboxylic acid, 2,4,6-triisopropylbenzenesulfonyl chloride, 2,4-dichlorophenoxyacetic acid, 2,5-dichlorobenzoic acid, 2,5-dimethoxyphenylacetic acid, 2,6-dichloro-5-fluoro-3-pyridinecarboxylic acid, 2,6-dichloronicotinic acid, 2,6-dichlorophenylacetic acid, 2,6-dichloropyridine-4-carboxylic acid, 2,6-dimethoxynicotinic acid, 2-[(2,6-dichloropyridin-4-yl)thio]acetic acid, 2-[[4-(trifluoromethyl)pyridin-3-yl]thio]acetic acid, 2-[1-(3-chlorobenzyl)-1H-indol-3yl]acetic acid, 2-[1-(6-chloro-3-pyridazinyl)-1H-indol-3-yl]acetic acid, 2-[1-(6-chloropyridazin-3-yl)-3,5-dimethyl-1H-pyrazol-4-yl]-5-methoxybenzoic acid, 2-amino-6-chloro-9h-purine-9-acetic acid, 2-aminobenzophenone-2′-carboxylic acid, 2-aminonicotinic acid, 2-benzofurancarboxylic acid, 2-benzylamino-benzoic acid, 2-bisbenzylcarboxylic acid, 2-bromo-5-methoxybenzoic acid, 2-bromobenzoic acid, 2-carboxymethyl-2H-benzotriazole, 2-carboxymethyl-4-methyl-1(2H)-phtalazinone, 2-chloro-5-(methylthio)benzoic acid, 2-chloro-6-(2-methoxyphenyl) nicotinic acid, 2-chloro-6-[4-(methylsulfanyl)phenyl] nicotinic acid, 2-chloro-6-fluorophenylacetic acid, 2-chloro-6-methoxyisonicotinic acid, 2-chloro-6-methylnicotinic acid, 2-chloro-6-thien-2-ylnicotinic acid, 2-chlorocirmamic acid, 2-chlorohippuric acid, 2-chloroisonicotinic acid, 2-fluoro-3-(trifluoromethyl)benzoic acid, 2-hydroxy-6-(trifluoromethyl)nicotinic acid, 2-iodofluorene-5-carboxylic acid, 2-methoxyphenoxyacetic acid, 2-methyl-1,8-naphthyridine-3-carboxylic acid, 2-methyl-2-(1H-1,2,4-triazol-1-yl)propanoic acid, 2-methyl-2-(7-methylindan-4-yloxy)propionic acid, 2-methyl-3-indoleacetic acid, 2-methyl-5-(trifluoromethyl)oxazole-4-carboxylic acid, 2-methyl-5-phenylfuran-3-carboxylic acid, 2-methylsulfonylbenzoic acid, 2-naphthalenesulfonyl chloride, 2-naphthylacetic acid, 2-nitro-5-thiocyanatobenzoic acid, 2-nitro-alpha,alpha,alpha-trifluro-p-toluic acid, 2-nitrophenylpyruvic acid, 2-norbornaneacetic acid (exo,endo, racemate), 2-oxo-6-pentyl-2h-pyran-3-carboxylic acid, 2-phenyl-1,3-thiazole-4-carboxylic acid, 2-thiophen-2-yl-quinoline-4-carboxylic acid, 3-(1,3-benzoxazol-2-ylsulfanyl)propionic acid, 3-(1H-1,2,3-benzotriazol-1-yl)propanoic acid, 3-(1H-indazol-1-yl)propionic acid, 3-(1H-tetrazol-1-yl) benzoic acid, 3-(2,3-dihydro-1,4-benzodioxin-6-yl)-1H-pyrazole-5carboxylic acid, 3-(2,4-dimethylbenzoyl)propionic acid, 3-(2,6-dichlorophenyl)-5-methylisoxazole-4-carboxylic acid, 3-(2-chlorophenyl)-5-isoxazole-carboxylic acid, 3-(2-chlorophenyl)-5-methylisoxazole-4-carboxylic acid, 3-(2-furyl)acrylic acid, 3-(2-methoxy-phenyl)-3-methyl-butyric acid, 3-(2-thienoyl)propionic acid, 3-(2-thioxo-benzooxazol-3-yl)-propionic acid, 3-(3,4-methylenedioxyphenyl)propionic acid, 3-(3-methyl-1H-pyrazol-1-yl)propanoic acid, 3-(3-methylindol-1-yl)propionic acid, 3-(4-bromo-3,5-dimethylpyrazol-1-ylmethyl)benzoic acid, 3-(4-chlorophenyl)-1H-pyrazole-5-carboxylic acid, 3-(4-chlorophenyl)-5-isoxazolecarboxylic acid, 3-(4-ethoxybenzoyl)propionic acid, 3-(4-hydroxyphenyl)-propionic acid, 3-(4-methylsulphonylbenzoyl)propionic acid, 3-(4-tert-butyl-phenyl)-acrylic acid, 3-(5-bromo-2-ethoxyphenyl)acrylic acid, 3-(cyclopentyloxy)-4-methoxybenzoic acid, 3-(methoxycarbonyl)-2,2,3-trimethylcyclopentane-1-carboxylic acid (racemate), 3-(methylsulfonyl)benzoic acid, 3-(trifluoromethylthio)benzoic acid, 3,4,5-trimethoxybenzoic acid, 3,4,5-trimethoxyphenylacetic acid, 3,4-dichloro-alpha-methoxyphenylactic acid (racemate), 3,4-dichlorobenzoic acid, 3,4-dichlorophenylacetic acid, 3,4-difluorohydrocinnamic acid, 3,4-dihydroxyhydrocinnamic acid, 3,4-dimethoxybenzenesulfonyl chloride, 3,5-diaminobenzoic acid, 3,5-dibromobenzoic acid, 3,5-dichloro-2,6-dimethoxybenzoic acid, 3,5-dichlorobenzoic acid, 3,5-diiodo-4-pyridone-1-acetic acid, 3,5-dimethoxybenzoic acid, 3,5-dimethylisoxazole-4-carboxylic acid, 3,5-di-tert-butylbenzoic acid, 3,6-dichlorobenzo[b]thiophene-2-carboxylic acid, 3-[(4-chlorobenzyl)oxy]-2-thiophenecarboxylic acid, 3-[1,2-dihydro-2-oxo-5-(trifluoromethyl)-pyrid-1-yl]propionic acid, 3-[2,3-dihydro-1-(1H)-indole]propanoic acid, 3-[3,4-(trimethylenedioxy)benzoyl]propionic acid, 3-acetamido-p-toluic acid, 3-acetoxycinnamic acid, 3-benzoyl-2-pyridinecarboxylic acid, 3-bromo-4-methylbenzoic acid, 3-chloro-4-hydroxyphenylacetic acid, 3-chloro-6-fluorobenzo[b]thiophene-2-carboxylic acid, 3-chlorobenzo[b]thiophene-2-carboxylic acid, 3-chlorocinnamic acid, 3-hydroxy-2-methyl-4-quinolinecarboxylic acid, 3-hydroxyadamantane-1-carboxylic acid, 3-imidazol-1-yl-propionic acid, 3-indolylacetic acid, 3-methoxy-4-nitrobenzoic acid, 3-phenoxybenzoic acid, 3-phenoxyphenylacetic acid, 3-phenyl-5-isoxazolecarboxylic acid, 3-phenylpropionic acid, 3-phthalimido-propionic acid, 3-tert-butyl-1-methylpyrazole-5-carboxylic acid, 3-tert-butyl-6-methylsalicylic acid, 3-thiopheneacetic acid, 4-(1H-tetrazol-1-yl)benzoic acid, 4-(3,4-ethylenedioxyphenyl)butyric acid, 4-(4-hydroxyphenyl)benzoic acid, 4-(4-methoxyphenyl)thiophene-2-carboxylic acid, 4-(4-methyl-piperdidine-1-sulfonyl)-benzoic acid, 4-(difluoromethoxy)benzoic acid, 4-(ethylthio)benzoic acid, 4-(methylsulfonyl)benzoic acid, 4-(morpholin-4-ylmethyl)benzoic acid, 4-(phenylthio)benzoic acid, 4-(trifluoroacetyl)benzoic acid, 4-(trifluoromethyl)hydrocinnamic acid, 4-(trifluoromethyl)phenylacetic acid, 4,7-dimethylpyrazolo(1,5-a)pyrimidine-3-carboxylic acid, 4-[(4-hydroxyphenyl)sulfonyl]benzoic acid, 4-acetamidobenzenesulfonyl chloride, 4-acetamidocinnamic acid, 4-amino-5-carboxy-2-ethyl-mercaptopyrimidine, 4-amino-5-chloro-2-methoxybenzoic acid, 4-aminobenzoic acid, 4-biphenylacetic acid, 4-bromo-3,5-dihydroxybenzoic acid, 4-bromocinnamic acid, 4-bromomandelic acid (racemate), 4-bromophenylacetic acid, 4-butoxyphenylacetic acid, 4-carboxy-1-(4-chlorobenzyl)pyrrolidin-2-one (racemate), 4-carboxybenzenesulfonamide, 4-carboxy-N-(fur-2-ylmethyl)pyrrolidin-2-one (racemate), 4-chloro-2-nitrobenzoic acid, 4-chloro-3-ethyl-2-methylquinoline-6-carboxylic acid, 4-chloro-o-anisic acid, 4-chlorophenoxyacetic acid, 4-chlorophenylacetic acid, 4-cyano-3,5-dimethyl-1H-pyrrole-2-carboxylic acid, 4-cyanobenzoic acid, 4-dimethylaminobenzoic acid, 4-fluoro-1-naphthoic acid, 4-hex-5-enyloxy-benzoic acid, 4-methoxybenzylidenecyanoacetic acid, 4-methyl-1,2,3-thiadiazole-5-carboxylic acid, 4-methyl-2-(2-pyridinyl)-1,3-thiazole-5-carboxylic acid, 4-methyl-2-(2-thienyl)-1,3-thiaxzole-5-carboxylic acid, 4-methyl-2-(3-pyridinyl)-1,3-thiazole-5-carboxylic acid, 4-methyl-2-phenyl-1,2,3-triazole-5-carboxylic acid, 4-nitrophenylacetic acid, 4-oxo-2-thioxo-3-thiazolidinylacetic -thiazolidinylacetic acid, 4-oxo-3,4-dihydro-phthalazine-1-carboxylic acid, 4-oxo-4-(4-propoxyphenyl)butanoic acid, 4-oxo4,5,6,7-tetrahydrobenzo[b]furan-3-carboxylic acid, 4-phenyl-1,2,3-thiadiazole-5-carboxylic acid, 4-tert-butylbenzenesulfonyl chloride, 4-vinylbenzoic acid, 5-(2-hydroxyethyl)-2-thiophenecarboxylic acid, 5-(2-nitrophenyl)-2-furoic acid, 5-(2-phenyleth-1-ynyl)-2-furoic acid, 5-(2-thienoyl)butyric acid, 5-(3-nitrophenyl)-2-furoic acid, 5-(4-chlorophenyl)-1H-pyrrole-2-carboxylic acid, 5-(4-chlorophenyl)-2-furoic acid, 5-(4-methoxyphenyl)-2-thiophenecarboxylic acid, 5-(methylthio)salicylic acid, 5,6-dichloronicotinic acid, 5-benzyloxyindole-3-acetic acid, 5-bromo-2-furoic acid, 5-bromonicotinic acid, 5-bromoorotic acid, 5-bromothiophene-2-carboxylic acid, 5-ethyl-2-indolecarboxylic acid, 5-fluoroindole-3-acetic acid, 5-hex-1-ynylnicotinic acid, 5-hydroxy-2,3-norbornanedicarboxylic acid gamma-lactone (racemate), 5-hydroxynicotinic acid, 5-methoxy-1-indanone-3-acetic acid (racemate), 5-methoxy-2-methyl-3-indoleacetic acid, 5-methyl-1-phenylpyrazole4-carboxylic acid, 5-methyl-3-phenylisoxazole-4-carboxylic acid, 5-methyl-4-(1H-1,2,4-triazol-1-ylmethyl)-2-furoic acid, 5-methyl-4-(morpholin-4-ylmethyl)-2-furoic acid, 6-(1H-pyrazol-1-yl)nicotinic acid, 6-(4-morpholinyl) acid, 6-(benzylsulfanyl)-2-pyrazinecarboxylic acid, 6-(ethylsulfanyl)-2-pyrazinecarboxylic acid, 6-bromocoumarin-3-carboxylic acid, 6-bromopicolinic acid, 6-chloro(2H)-1-benzopyran-3-carboxylic acid, 6-hydroxy-2-methylquinoline-4-carboxylic acid, 6-hydroxy-2-naphthoic acid, 6-methylchromone-2-carboxylic acid, 6-N-butyl-2-(3,4-dimethoxyphenyl)-8-methylquinoline-4-carboxylic acid, 6-oxo-1,4,5,6-tetrahydropyridazin-3-carboxylic acid, 6-oxo-1-[4-(trifluoromethyl)benzyl]-1,6-dihydro-3-pyridinecarboxylic acid, 6-phenylhexanoic acid, 7-carboxymethoxy-4-methylcoumarin, 7-chlorolcynurenic acid, 7-ethoxybenzofuran-2-carboxylic acid, 7-methoxybenzofuraN-2-carboxylic acid, 7-methoxycoumarin-4-acetic acid, 8-hydroxyquinoline-2-carboxylic acid, 8-methoxy-1,2,3,4-tetrahydronaphthalene-2-carboxylic acid (racemate), 9-fluorenone4-carboxylic acid, acemetacin, acetic acid, alpha-(ortho-tolyl)-cyclohexaneacetic acid (racemate), benzenesulfonyl chloride, benzo[b]thiophene-3-acetic acid, benzo[c]furaN-2-carboxylic acid, benzoic acid, benzoyl-DL-leucine, beta-(naphthylnercapto)acetic acid, Boc-L-hydroxyproline, bumetanide, chloramben, cis-pinonic acid (racemate), coumalic acid, coumarin-3-carboxylic acid, cycloheptanecarboxylic acid, cyclohexanepropionic acid, cyclohexylidenecyanoacetic acid, cyclopentanecarboxylic acid, D-camphor-10-sulfonyl chloride, DL-3,4-dihydroxymandelic acid, DL-indole-3-lactic acid, DL-thioctic acid, fenbufen, flufenamic acid, fluorene-9-acetic acid, hydantoic acid, imidazo[2,1-b]benzothiazole-2-carboxylic acid, indole-3-glyoxylic acid, indole-6-carboxylic acid, indomethacin, indoprofen (racemate), isoquinoline-3-carboxylic acid hydrate, kynurenic acid, 1-2-oxothiazolidine-4-carboxylic acid, levulinic acid, mefenamic acid, N-(1-naphthyl)maleamic acid, N-(2,4,6-trimethylphenyl)maleamic acid, N-(2-cyanoacetyl)anthranilic acid, N-(3-methoxyphenyl)maleamic acid, N,N-diethyl-3,6-difluorophthalamic acid, N-acetyl-L-tyrosine, naproxen, N-benzoyl-D-alanine, N-carbamyl-L-tryptophan, N-formyl-2-phenylalanine, N-formyl-dl-phenylalanine, nicotinic acid, niflumic acid, N-phenethylmaleamic acid, N-phthaloyl-DL-alpha-amninobutyric acid (racemate), N-tosyl-3-pyrrolecarboxylic acid, o-(3-carboxybenzyl)-4-chloroacetophenone oxime, o-benzamidoglycolic acid, phthalide-3-acetic acid (racemate), pyrazinecarboxylic acid, quinoline-3-carboxylic acid, quinoline-6-carboxylic acid, S-(−)-2-[(phenylamino)carbonyloxy]propionic acid, S-(+)-ibuprofen, s-(thiobenzoyl)thioglycolic acid, S-benzylthioglycolic acid, suprofen (racemate), tetrahydro-2-furoic acid (racemate), thymine-1-acetic acid, trans-1-methyl-4-carboxy-5-(3-pyridyl)-2-pyrrolidinone (racemate), trans-2,5-difluorocinnamic acid, trans-2-chloro-6-fluorocinnamic acid, trans-2-phenylcyclopropane-1-carboxylic acid (racemate), -trans-3,4-methylenedioxycinnamic acid, trans4-chloro-3-nitrocinnamic acid, xanthene-9-carboxylic acid, Z-beta-alanine.

The following abbreviations of protective groups for peptide synthesis have been used in the list of the preferred and non-limiting examples for reactants Lᵣ² and Lᵣ¹:

| | |
|---|---|
| 2-Br-Z | 2-bromobenzyloxycarbonyl |
| 2,6-Cl2-Bzl | 2,6-dichlorobenzyl |
| 4-methyl-bzl | 4-methylbenzyl |
| Acm | acetamidomethyl |
| All | allylether |
| Alloc | allyloxycarbonyl |
| Bzl | benzyl |
| Boc | tert.-butyloxycarbonyl |
| Dde | (4,4-dimethyl-2,6-dioxocyclohex-1-ylidene)-ethyl |
| Fmoc | 9-fluorenylmethyloxycarbonyl |
| ivDde | (4,4-dimethyl-2,6-dioxocyclohex-1-ylidene)-3-methylbutyl |
| Mts | mesitylene-2-sulfonyl |
| NO2 | nitro |
| O2 | sulfone |
| OAll | allylester |
| OBzl | benzylester |
| OCHx | alkylester |
| OMe | methylester |
| OtBu | tert.-Butylester |
| Pbf | Pentafluorophenyl |
| tBu | tert.-Butyl |
| Tos | p-toluolsolfonyl |
| Trt | Triphenylmethyl/Trityl |
| Z | benzyloxycarbonyl |

For the synthesis of ligands of formula (5), solid phase synthetic methods are preferred. Here, the compound under construction is covalently attached to an insoluble solid support throughout the solid-phase synthesis. Preferably, the bond between the synthesis phase and the ligand is achieved via a linker that can be cleaved under specific, gentle conditions with an appropriate reagent to yield the desired compound.

Moreover, the ligands forming the library are preferably provided by combinatorial methods. As a consequence, solid phase combinatorial synthesis is particularly preferred for the provision of these compounds.

In one embodiment of this synthesis strategy, a first reactant (e.g. Lᵣ²) is bound to the solid phase used in synthesis, normally via a bivalent linking moiety (“linker”). After connecting the second reactant (e.g. Lᵣ¹), the ligand can be cleaved from the solid phase due to the presence of the linker under gentle conditions with an appropriate reagent. After cleavage, the linker as a whole or parts of it may remain attached to the ligand which then comprises the following structure:

L¹-L²-Ln (5a)

wherein L¹ and L² are as defined above and the optional group Ln is the part of the linker remaining attached to the ligand after its cleavage from the solid phase. It should be understood that during the cleavage reaction, the linker or its parts may be chemically modified. Suitable linkers as well as reactions for their cleavage and resulting groups Ln are well established in the art of solid phase synthesis. Suitable examples which are also applicable for the synthesis of the present ligands are described, for example, in WO 00/73796 in the context of solid phase synthesis of anchors and ligand anchor conjugates. Preferably, the linker and the cleaving reaction is selected such that the ligand L¹-L² is released either with the unprotected functional group of L² which is required for the immobilization of the ligand L¹-L² on the support used in the screening step or with the unprotected functional group of Ln of the ligand L¹-L²-Ln which is required for the immobilization of the ligand L¹-L²-Ln on the support used in the screening step.

A preferred process for the a solid phase synthesis of a ligand thus comprises the steps of:

a) covalently binding a linker to a solid phase, then

b) binding an amino acid with a protected amino group as a first reactant Lᵣ² to the linker to yield the building block L² connected to the linker,

c) selectively removing the protecting group of the amino group of L²,

d) coupling a carboxylic or sulphonic acid as a second reactant Lᵣ¹ to the amino group of L² under formation of an amide or sulphonamide bond,

e) cleaving the linker-ligand conjugate or the ligand from the solid phase to release the ligand or the ligand-linker conjugate.

The building block connected with the linker in step b) can contain additional protective groups if necessary for the synthesis of the ligand, Ligand-Tag or ligand anchor conjugate. Protective groups for functional groups and their applications are known by one with ordinary skill in the art can be used for the, preferred process of solid phase synthesis for fulfilling different functions:

as temporary protecting group for the amino group of L2 to avoid side reactions during step b) and to be removed in step c),

as orthogonal side chain protecting group to be removed after step c) and before the step c) (contacting a target of interest with a said binding matrix) of the inventive screening method,

as integral part of the building block L1 and/or L2 introduced not to be removed at all.

Information on the types and applications of protective groups are described for example in,,Protective Groups in Organic Synthesis, Theodora W. Greene and Peter G. M. Wutz, third Edition, Wiley Interscience. Preferably, an Fmoc-group is used as protecting group for the amino group of L² in this process.

Together with step e) optionally protective groups of the ligands can be removed. In addition further protective groups can be removed before the step of the immobilization of a ligand on the support used for screening.

The ligands comprising structures of formulae (5) or (5a) are preferably immobilised directly or via anchors on a spatially addressable screening array so that each array field presents another scaffold-free combination of Lᵣ¹ and Lᵣ² in the ligand L¹-L², i.e. the reactants are direcly combined to yield the ligand. If the ligands are attached to activated anchors, such as those of formula (1), already immobilized on the support used for screening, care should be taken during their synthesis to introduce suitable functional groups (e.g. a thiol group).

For the purpose of binding the ligand to an anchor molecule, in particular an activated anchor molecule (e.g. of formula (1)) already immobilised on the support used for screening, it is preferred that the ligands are supplied with a specific structure (“ligand-tag”).

The structure of such a ligand-tag may be depicted by the formula:

Z-A-Y, (6)

wherein

A is a chemical bond or a hydrocarbon chain of 2 to 50, preferably 5 to 30 C-atoms, optionally interrupted by heteroatoms, amide or ester bonds,

Y is a functional group to react with the ligand, and

Z is a functional group which is able to react with the head group of (the) a corresponding anchor molecule, preferably a thiol, carboxyl or amino group. Particularly preferred is a thiol, capable of reacting with a mercaptophilic head group of the anchor molecules as described above.

Preferably, A is unbranched to minimise unspecific interactions between the “ligand tag” and the target. Heteroatoms suitable for A comprise O, N, S, Si, P, B.

Preferred are groups A of the general formula:

ti —(CH₂)ₗ-Q⁶-[(CH₂)ₘ-Q⁷]ₙ-(CH₂)ₒ-Q⁸-[(CH₂)_{m′}-Q ⁹]_{n′}-(CH₂)_{o′}-Q¹⁰-(CH₂)_{p′}- (7)

wherein the variables, independently of each other, are defined as follows and numerical ranges are to comprise their respective limiting values as well as all integers in-between:

| | |
|---|---|
| Q⁶ to Q¹⁰ | represent independently —NH—C(O)—, —C(O)—NH—, —NH—C(O)—O−, —O—C(O)—HN—, |
| | —C(O)—O—, —O−C(O)—, a heteroatom or a bond; |
| 1, p,p′ | are independently integers from 0 to 5, preferably 0 to 3; |
| m, m′, o, o′ | are independently integers from 1 to 5, preferably 1 to 3, particularly preferably |
| | 2; |
| n,n′ | are independently integers from 0 to 20, preferably 2 to 15 and particular |
| | preferably 3 to 10, with the proviso that at least one of n and n′ is not 0. |

More preferably, A comprises at least 1 amide bond and at least 4 heteroatoms. Particularly preferred is an A comprising two amide bonds and four oxygen atoms.

Examples for Y are primary and secondary amino groups, carboxylic acid groups, hydroxyl groups, hydroxylamino groups, ester, aldehyde and other carbonyl moieties. Preferably, Y is —NH₂, —NHR⁵, —NR⁵OH, —C(O)H, —C(O)OR⁵, or —C(O)OH, wherein R⁵ is a C1-C6 alkyl group such as methyl, ethyl, n-propyl, i-propyl etc.. Most preferably, Y is a primary amino group. However, many chemical reactions can be used to bind the ligand-tag Z-A-Y to the ligand and to the anchor molecule so the provided examples are not limiting. One skilled in the art can extend the list of examples and knows the chemical reactions like addition reactions, substitution reaction and condensation reactions leading to the desired chemical bond with the ligand.

The selection of the optimum ligand-tag for the inventive method depends on the ability of the ligand-tag to (a) minimise unspecific binding of the target to the ligand-tag, (b) present the ligand in a suitable distance from the SAM to the target to avoid steric repulsion between the SAM and the target and (c) provide a high mobility of the ligand for optimum binding capability. The selection of the ligand-tag also depends on the size and chemical nature of the target. Such ligand-tags, if used, are either directly attached to the ligand during its synthesis or immediately prior to its coupling with the anchor molecule. In a preferred embodiment, each immobilised ligand possesses the same ligand-tag.

With the ligand being covalently bound to the functional group Y of the ligand-tag, there is provided a ligand/ligand-tag conjugate which can be immobilised on the support used for screening. While a direct immobilisation is possible, the ligand/ligand-tag conjugates are preferably chosen as to provide suitable functional groups Z reacting to form a covalent bond with the activated head group of an anchor structure already present on the respective support. Preferred ligand/ligand-tag conjugates are those of the structure

Z-A-Y′-L, (8)

wherein Z and A are defined as in formula (6), and Y′ is a moiety such as an amide or ester bond resulting from the reaction of any of the above groups Y with a corresponding functional group of the ligand.. For example, Y′ represents —NHC(O)—, —C(O)NH—, —C(O)O—, or —OC(O)—.

The structure of the ligand L varies depending on the target structure. However, in order to be able to form the above bond Y′ with the ligand-tag, L is usually provided by a molecule having at least one functional group capable of reacting with Y of the ligand-tag, such as an alcohol, a primary or secondary amine, a carboxylic acid, a carboxylic acid ester, an aldehyde or another carbonyl compound. Apart from this functional group, the structure of the ligand is chosen following the criteria set out above.

In accordance with preferred embodiments defined for the ligands and the ligand-tags used in the present invention, particularly preferred ligand/ligand-tag conjugates of the present invention correspond to the formula

Z-A-HNC(O)-L²-L¹ (9)

wherein Z and A are as defined in formula (6),

L² is an amino acid residue as defined as a preferred embodiment of formula (5), which uses its carboxylic group to form an amide bond with the ligand-tag and its amino group to form a amide or sulfonamide bond with L¹, and

L¹ is a building block with a carboxylic acid group or sulfonic acid group function, using its functional group to complete the amide or sulfonamide bond, equally as defined as a preferred embodiment of formula (5).

As outlined before, it is advantageous that the ligands are assembled in an array format and that a plurality of ligands is immobilized on a chip. Therefore, in a preferred embodiment, the invention relates to a plurality of ligand/ligand-tag conjugates immobilized on the solid support used for screening in the form of an array, more preferably an array comprising at least 1536, 3072, 4608 or 9216 different types of ligands.

Furthermore, the invention relates to a screening chip (binding matrix) comprising the above array, wherein, preferably, the ligands are immobilized via anchor molecules forming a self assembled monolayer preferably comprising additionally dilution molecules.

Just as the ligands, ligand/ligand-tag conjugates are preferably synthesised via combinatorial solid phase synthesis. For solid phase synthesis of the ligands provided with a ligand-tag it is preferred to first immobilise the ligand tag at the solid support and subsequently connect first L² and then L¹ to the ligand tag. Of course, the ligand-tag can be directly synthesised at the solid phase, followed by combinatorial synthesis of the actual ligand structure comprising L² and L¹ as described above. In both cases, the ligand tag may be covalently bound to the solid phase directly or via a linker. With respect to suitable linkers for the solid phase synthesis of a ligand/ligand-tag, the information given above with respect to solid phase synthesis of the ligands alone applies.

In order to connect the ligand tag and the ligand, the functional group Y of formula (6) is reacted with a suitable functional group of Lᵣ², preferably the one which is described above as serving for the immobilisation of the ligand on the solid support used for screening, e.g. an amino group, a hydroxyl group, a thiol, a carboxylic acid, a sulfonic acid. In preferred cases, where Lᵣ² is an amino acid, its carboxylic group can be used for this purpose. In this case, Y preferably represents an amine to form an amide bond with Lᵣ²

Should the linker and the ligand tag be coupled prior to their attachment to the solid phase to yield a linker/ligand tag conjugate of the following formula:

Ln-Z-A-Y (10)

it can be useful to mask Y with a protecting group to avoid side reactions while covalently binding the conjugate to the solid phase. The protecting group is removed afterwards, before the synthesis of the ligand structure is started.

A preferred linker for the solid phase synthetic methods described herein is 3-(4-(diphenylmethyl-phenoxy-)butyric acid, introduced as 3-(4-(diphenylhydroxymethyl-phenoxy-)butyric acid as illustrated in the preparative example in step 7 of the synthesis of a ligand-tag/linker conjugate Ln-Z-A-Y-Fmoc where Y is a amino group protected with the Fmoc-protecting group:

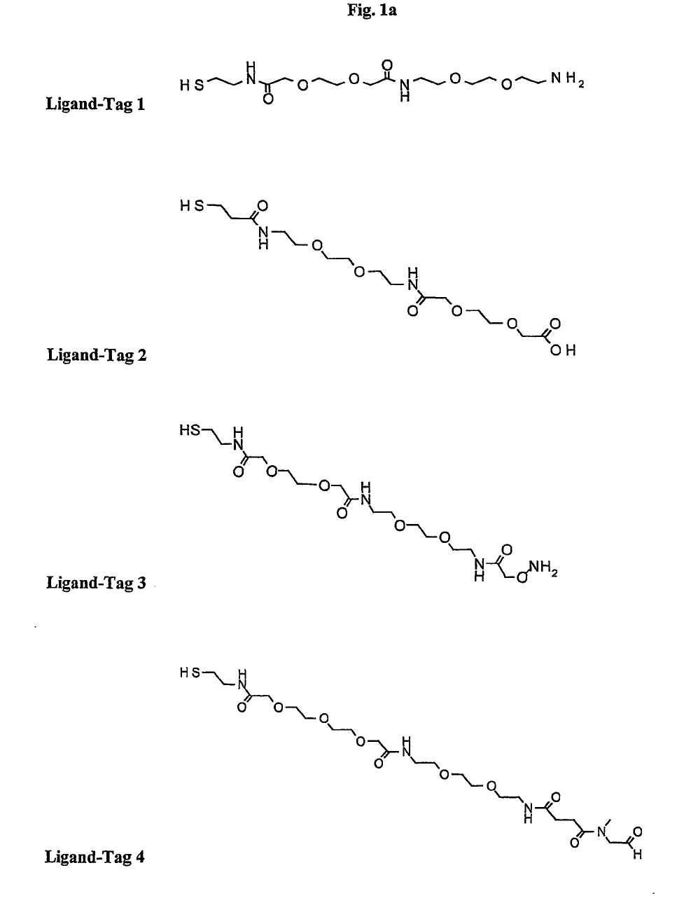

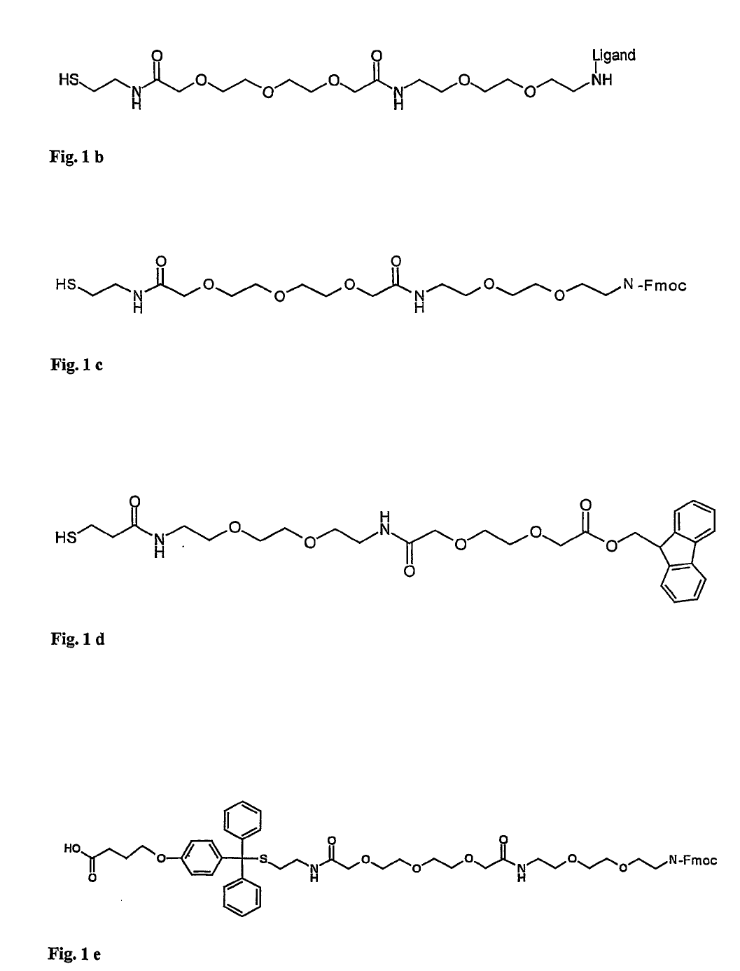

Examples of preferred ligand-tags are shown in FIG. 1a where Ligand-Tag 1 is most preferred. Ligand-Tag 1 can be synthesised from the reaction product of step 6 of the synthesis of the ligand-tag/linker conjugate X-Z-A-Y-Fmoc described in the examples. Ligand-Tag 2 can be synthesised respectively by deprotecting the reaction product of step 7 of the synthesis of the ligand-tag/linker conjugate X-Z-A-Y-Fm described in the examples.

An example for a ligand supplied with a ligand tag is given in FIG. 1b:

An example for a preferred Y-masked ligand tag Z-A-Y-Fmoc with the Fmoc protecting group (9-fluorenylmethyloxycarbonyl) is given in FIG. 1c

An example for a preferred Y-masked ligand tags Z-A-Y-Fm with the Fm protecting group (9-fluorenylmethylester) for a carboxylic group is given in FIG. 1d:

An example for a preferred ligand-tag/linker conjugate Ln-Z-A-Y-Fmoc with Y protected with the Fmoc-protecting group is given in FIG. 1e.

In a preferred embodiment, an array and screening chip (binding matrix) according to the present invention comprise ligands L¹-L² attached with a ligand tag Z-A-Y (6), more preferred is the ligand/ligand-tag conjugate represented by the formula Z-A-HN-L²-L¹, wherein Z, A and Y are defined as above.

Accordingly, a preferred process for the generation of ligands carrying ligand tags using solid ,phase synthesis comprises the steps of:

a1) covalently binding a linker to a solid phase and coupling a ligand tag with the linker or

a2) coupling a linker and a ligand-tag and covalently binding them via the linker to the solid phase

b) binding an amino acid with a protected amino group as a first reactant Lᵣ² to the ligand tag to yield the building block Lᵣ²,

c) selectively removing the protecting group of the amino group of Lᵣ²,

d) coupling a carboxylic or sulphonic acid as a second reactant Lᵣ¹ to the amino group of L² under formation of an amide or sulphonamide bond,

e) cleaving the ligand/ligand-tag conjugate, optionally carrying parts of the linker used in (a1 or a2) from the solid phase to release the ligand/ligand tag conjugate.

The building block connected with the linker in step b) can contain additional protective groups if necessary for the synthesis of the ligand, ligand-tag or ligand anchor conjungate. Protective groups for functional groups and their applications are known by one with ordinary skill in the art can be used for the preferred process of solid phase synthesis for fulfilling different functions:

as temporary protecting group for the amino group of L2 to avoid side reactions during step b) and to be removed in step c),

as orthogonal side chain protecting group to be removed after step c) and before the step c) (contacting a target of interest with a said binding matrix) of the inventive screening method,

as integral part of the building block L1 and/or L2 introduced not to be removed at all.

Information on the types and applications of protective groups are described for example in,,Protective Groups in Organic Synthesis, Theodora W. Greene and Peter G. M. Wutz, third Edition, Wiley Interscience. Preferably, an Fmoc-group is used as protecting group for the amino group of L² in this process.

Together with step e) optionally protective groups of the ligands can be removed. In addition further protective groups can be removed before the step of the immobilization of a ligand on the support used for screening.

The final ligand/ligand-tag conjugates are preferably cleaved from the synthesis support and immobilised on the solid support used for the screening step. This brings about the additional advantage that the compounds from one synthesis plate (synthesis support) can be used for up to 1000 screening plates (solid support used for the screening). Preferably, the ligand/ligand-tag conjugates are contacted with the activated anchors already immobilised on the support used for screening to allow the functional group Y of the ligand tag to react with the head group of the anchor for the formation of a covalent bond.

If desired, a similar process as the one above can equally be applied if ready made ligand-anchor conjugates are integrally immobilized to form the binding matrix for the method of the invention. In this case, a preferred synthesis of a ligand anchor conjugate comprises the steps of:

a′) covalently binding a linker to a solid phase and synthesizing an anchor structure bound to the linker, then

b′) binding an amino acid with a protected amino group as a first reactant Lᵣ² to the anchor to yield L²,

c′) selectively removing the Fmoc-protecting group of the amino group of L², coupling a carboxylic or sulphonic acid as a second reactant Lᵣ¹ to the amino group of L² under formation of an amide or sulphonamide bond,

d′) cleaving the ligand anchor conjugate, optionally carrying parts of the linker used in (a) from the solid phase to release the ligand anchor conjugate or the ligand-linker conjugate.

Suitable conditions and reagents to be used in step (a′) are described in WO 00/73796. However, for reasons set out above, a step-wise immobilisation of anchors and ligands or ligand/ligand-tags is preferred for the purpose of the present invention.

Parallel high throughput screening of the library of candidate target binding molecules with the target molecule might lead to the identification of a subset of ligands. Preferred targets are proteins, DNA, RNA, oligonucleotides, prosthetic groups, vitamins, lipids, oligo- or polysaccharides, but also synthetic molecules, such as fusion proteins or synthetic primers. Particularly preferred are proteins, such as a protease.

The choice of the mode of detection is an important element in surface-based techniques for the screening of binding interactions. Suitable labelling methods for the detection of target-ligand interactions on a solid surface are radio-immunoassays and optical methods, as for example fluorescence or luminescence measurements (especially enzyme assays). In a preferred embodiment, the so-called ELISA technique (enzyme-linked immunosorbent assay), an immunoassay on solid phase, is used. Here, the solid support is used solely for the immobilisation of one interaction partner.

However, labels used in these approaches may have the disadvantage of influencing specific binding interactions. Besides, labelling requires extra synthesis and isolation steps. Considering the many new proteins that are or will be delivered from the isolation or expression of human genes, the possibility of label-free detection of interactions with small amounts of protein sample is desirable (see Haake et al. (2000), J. Anal. Chem. 366, 576-585). Suitable methods for the label-free detection of target-ligand interactions are reflection optical techniques. Reflection-optical methods comprise surface plasmon resonance (SPR) and reflective interference spectroscopy (RIfS). In these methods, the solid support is an integral part of the sensor system.

Surface plasmon resonance (SPR) detects changes in refractive index that occur at the transducer surface during the binding event under investigation. In this method, an optical support (preferably a prism) is covered with a thin metal film and the change in intensity of the intern at the prism reflected light that occurs upon ligand-target binding is measured as function of the wavelength or as function of the adjusted angle. The SPR method has proven to be very useful in various fields and is now an established technique. Therefore, it should be possible to explore new areas of application such as high-throughput screening (HTS).

Reflective interference spectroscopy (RIfS) is capable of using the partial reflection of light at interfaces for detecting changes in layer thickness. The attachment of biomolecules to binding partners (ligands) causes a shift in the intensity profile as a function of the wavelength. The shift of the detected curves is proportional to the change in layer thickness.

Another label-free method are biosensors based on quartz micro balances. The bonds between targets and ligands are measured by means of the weight increase affecting the frequency of oscillating quartz crystals (Ebara and Okahata, JACS 2000, 116: 11209-12).

However, in a preferred embodiment, the detection technique for ligand-target interaction during the method of the present invention is surface plasmon resonance (SPR).

After screening a combinatorial library for affinity towards a (protein) target certain thresholds have to be defined in order to select “hits”, i.e. molecules which bind to the target. In a preferred embodiment, hits are selected by ranking the molecules pursuant to their binding values. Each hit shows a binding value which is significant higher (preferably 2 fold, more preferred 4 fold higher and particular preferred 10 fold higher) than the average binding value for unspecific ligand-target interactions (noise level). Hit identification and selection can be supported by a software program (e.g. Jarray) which is able to determine and visualise the noise level by application of statistical methods.

Jarray is a Java-based software program for processing and visualising data from a database and in particular the data obtained for the binding values of the respective ligands in step d) of the screening method of the present invention that supports the identification and selection of a subset of specific binding molecules.

Jarray comprises a data base storing a plurality of records, a main processing system, a user input device and a display. The data from the database are visualised in a x, y-table on the screen or any other suitable medium.

If Jarray is used, ligands forming the library of step (a) of the method of the present invention, are preferably formed via binary combinatorial synthesis, starting from two sets of reactants. Particularly preferred are ligands comprising a structure of the above formula (5). The x co-ordinates (rows) represent the first set of building blocks, e.g. L² and the y co-ordinates (columns) represent the second set of building blocks, e.g. L¹ for the binary combinatorial synthesis.

Thus, each cell of the table (x, y co-ordinate) represents a member compound of the library screened according to the method of the present invention. If the binding value obtained for each ligand in step (d) above is visualised in a colour resolved manner, e. g. with darker shades representing a higher binding value, columns or rows of specific shades allow a conclusion on particularly active starting substances/molecular subunits present in the ligands of the library. At the same time, synergistic or antagonistic effects with respect to the target between the reagents comprised in the two sets of reagents used are visualised by particularly light or deeply coloured cells in rows or columns which otherwise have a comparably uniform appearance.

Most drug discovery development is performed through a series of optimisation cycles within a focused screening in order to meet a set of predetermined criteria for a drug candidate. The interpretation of the data resulting from the screening of a first library drives the new (second, tertiary, etc.) library design, creating an iterative cycle of combinatorial library synthesis and biological evaluation. One challenge in lead optimisation is to capture the data and to build structure-activity relationship (SAR) and quantitative structure-activity relationship (QSAR) models.

The structural information obtained by the analysis of identified hits can be used to design a library of a more limited size with close structural resemblance to the original lead structure (so-called focused library). The library for a focused screen preferably comprises more than about 10, particularly more than 100, more particularly more than 1000 compounds.

One approach favours using structural motifs, which have distinguished themselves by appearing frequently in identified hits, so-called “privileged structures” which can be identified easily by Jarray because of highlighted rows or columns (see above). Another approach is to incorporate key recognition elements for target binding (pharmacophoric patterns) that are relevant to the particular target under investigation.

Different computational methodologies are available to design such a focused library. Cell-based methods have been shown to be very effective for large-scale diversity problems, as demonstrated e.g. by successes with the DiverseSolutions method distributed by Tripos (Pearlman and Smith, 1998).

Methods that focus on monomers identify privileged building blocks based on hits obtained by screening a library. Different schemes were introduced such as monomer frequency count (Zheng W. et al., J. Chem. Inf. Comput. Sci. 1998, 38: 251-258) or rule-based monomer selection (Bravi G. et al., J. Chem. Inf. Comput. Sci. 2000, 40: 1441-1448). In each case, a new library is constructed by reusing new combinations of identified monomers.

Recently, artificial neural networks and evolutionary methods, such as genetic algorithms, have gained popularity in combinatorial library design and applications are expected to grow in the future (Weber L., Drug Discovery Today 1998, 3: 379-385; Zupan J. and Gasteiger J., 1999, ‘Neural networks in chemistry and drug design’, Wiley-VCH; Bohm H. J. and Schneider G., 2000, ‘Virtual screening for bioactive molecules’, Wiley-VCH). These self-learning techniques rely on training data sets from which fitness functions are derived that are used to guide the algorithms towards the design objective. In the case of structure-based focusing structural characteristics together with experimental data on bioactivity of compounds are used to establish the definition of fitness. Large datasets with a wide range of activities obtained under identical conditions are an ideal starting point for setting up an algorithm.

Our screening method is able to provide such data by covering a high-dynamic range of affinities under identical conditions. Moreover, affinity based screening selects compounds based on a single underlying property i.e. binding to a target. Thus, the outcome of an affinity-based screen of a combinatorial library lends itself to be used for feeding the above mentioned computational tools.

The final step is the biological evaluation of promising hits detected by solid phase screening in order to identify a drug lead compound that inhibits or activates the target molecule. A multitude of specific biological assays has been developed for this purpose (Hill D. C., Current Opinion in Drug Discovery and Development 1998, 1: 92-97; Nakayama G. R, Current Opinion in Drug Discovery and Development 1998, 1: 85-91). The screening method of the library of drug like molecules is preferably an in-vitro test in solution, i.e. a functional assay. Preferably, drug lead compounds bind with a K_{D} of less than micromolar to the active site of the target molecule.

### EXAMPLES

### Synthesis of Ligand-Tag Linker Conjungates

Synthesis of a ligand-tag/linker conjugate X-Z-A-Y-Fm where Y is a carboxylic group protected with the Fm-protecting group:

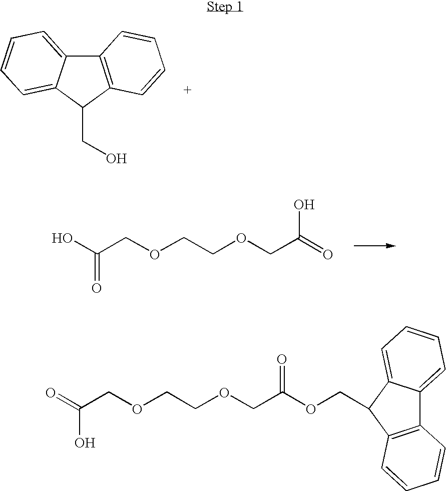

15 mg DMAP (dimethylaminopyridine) was added as a catalyst to a solution of 21.5 g 3,6-dioxaoctanic dicarboxylic acid (121 mmol) in 100 ml DCM (dichlormethane) and the mixture was cooled down to −10° C. Then 9.3 g DCC (dicyclohexylcarbodiimide) (46 mmol) dissolved in DCM was added and the solution stirred for 20 minutes. Then 26.22 g diisopropylethylamine DIEA (203 mmol) and the solution of 7.96 g 9-fluorenylmethanol (41 mmol) in DCM was added and the reaction mixture was stirred over night. The precipitate was removed by filtration and the solvent of the filtrate was evaporated under reduced pressure. The residue was dissolved in ethyl acetate and was washed three times with 1M hydrochloric acid. The organic phase was dried with sodium sulphate over night and then the solvent was evaporated under reduced pressure leading to an yellowish oil (13.5 g; yield=93.4%).

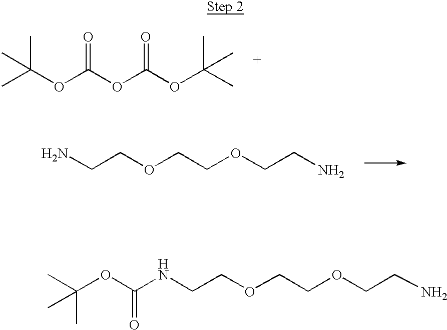

A solution of 19.6 g di-tert-butyl-dicarbonate (90 mmol) dissolved in 100 ml DCM was added slowly to a solution of 40.0 g 1,8-diamino-3,6-dioxaoctane (270 mmol) in 200 ml DCM. After stirring the reaction mixture over night the solvent was evaporated under reduced pressure. The residue was dissolved in ethyl acetate and was washed three times with 10% Na₂CO₃-solution. The organic phase was dried with sodium sulfate. Then the solvent was evaporated under reduced pressure. The solid residue (19.3 g; yield=86.4%, (relative to di-tert-butyl-dicarbonate)) was used in the next step without further purification.

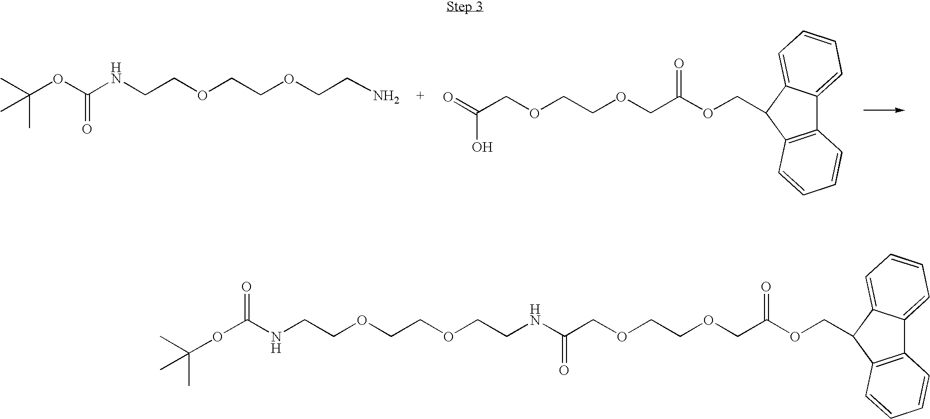

The solution of 13.5 g of the product from step 1 (38 mmol) and 4.6 mg of DMAP in 80 ml DCM was cooled down to −10° C. At this temperature a solution of 8.6 g DCC (42 mmol) in DCM was added slowly. After stirring the mixture for 20 minutes the solution of 10.3 g of the product from step 2 (42 mmol) in DCM was added slowly. The resulting mixture was stirred over night. The precipitate was collected by filtration and the DCM removed from the filtrate. The residue of the filtrate was dissolved in ethyl acetate and washed two times with 1M hydrochloric acid. The organic phase was dried with sodium sulphate over night and the solvent was evaporated under reduced pressure. The resulting product (19.0 g; yield=85%) was purified on a silica chromatography column using 300 g silica and subsequently the following eluents: a) 2.0 l ethyl acetate; b) 1.5 l ethyl acetate/methanol (90:10); 1.5 l ethyl acetate/methanol (80:20); c) 1.5 l ethyl acetate/methanol (50:50). After purification the product yield was 6.1 g=27.6%.

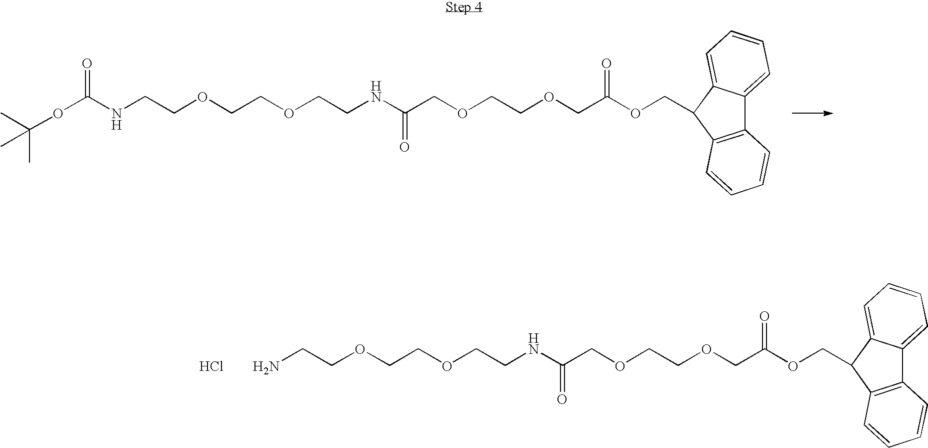

To the solution of 6.1 g product from step 3 (10.4 mmol) in DCM 4M hydrochloric acid in dioxane was added and the reaction was monitored by thin layer chromatography. After the end of the reaction the solvents were removed under reduced pressure resulting 6.7 g of a white solid (yield=99%).

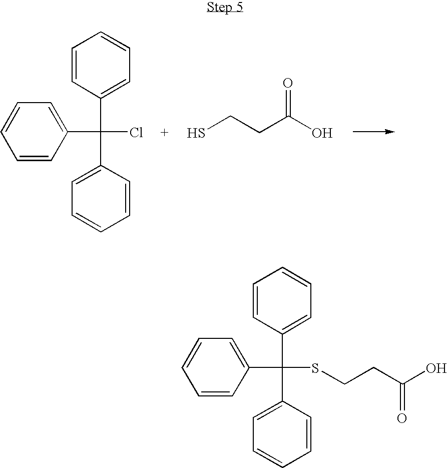

27.8 g triphenylmethylchloride (100 mmol) and 12.7 g 3-mercaptopropionic acid (12 mmol) were dissolved in 200 ml N,N-dimethylformamide. Then 40 ml pyridine (500 mmol) was added slowly and the reaction mixture was stirred over night. Then the solvent was removed under reduced pressure. The product was dissolved in ethyl acetate and washed two times with 1M hydrochloric acid. The organic phase was dried with sodium sulphate over night. The solvent was evaporated and the product crystallised from a solution of ethyl acetate and methanol to yield 22.9 g (yield=66%) of a white amorphous powder.

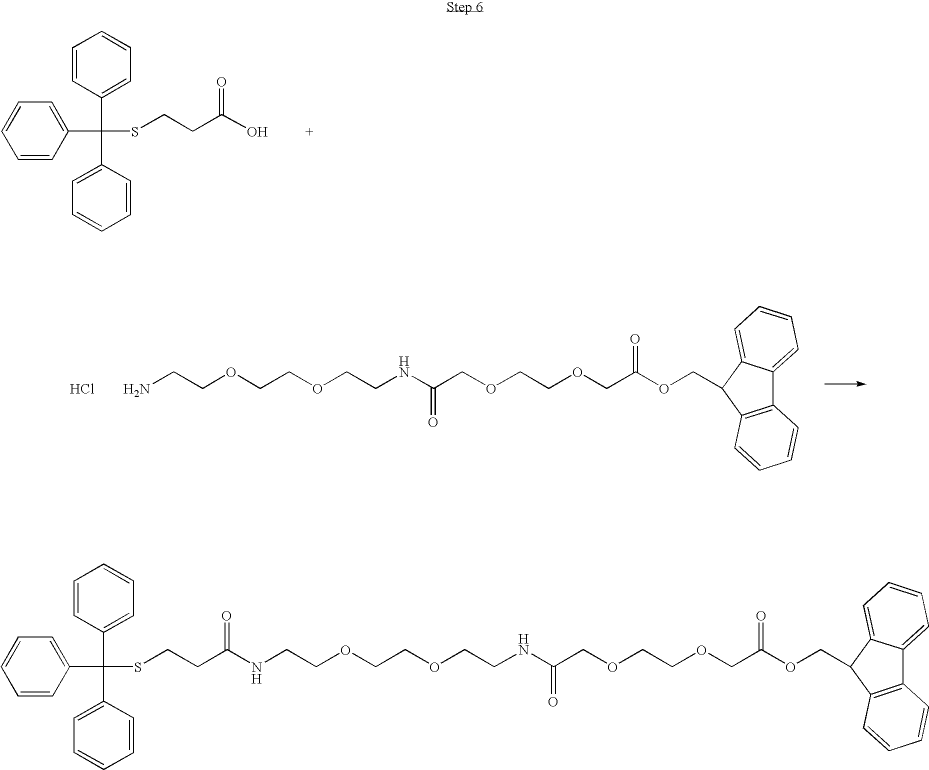

4.5 g of the product of step 5 (13 mmol) was dissolved in 150 ml tetrahydrofurane (THF). Then 2.6 g 1,1′-carbonyldiimidazole (16 mmol) was added to the mixture in small portions and the mixture was stirred for one more hour. Then a solution prepared by first dissolving 6.7 g of the product of step 4 (13 mmol) in 100 ml THF and then adding 1.7 g Diisopropylethylamine (13 mmol), was added to the reaction mixture. Then the reaction mixture was stirred over night. Then the solvent was removed under reduced pressure. The residue was dissolved in ethyl acetate and washed two times with 1M hydrochloric acid. The organic phase was dried with sodium sulphate over night. The solvent was evaporated under reduced pressure. The raw product (6.9 g; yield=65.8%) was used in the next step without further purification.

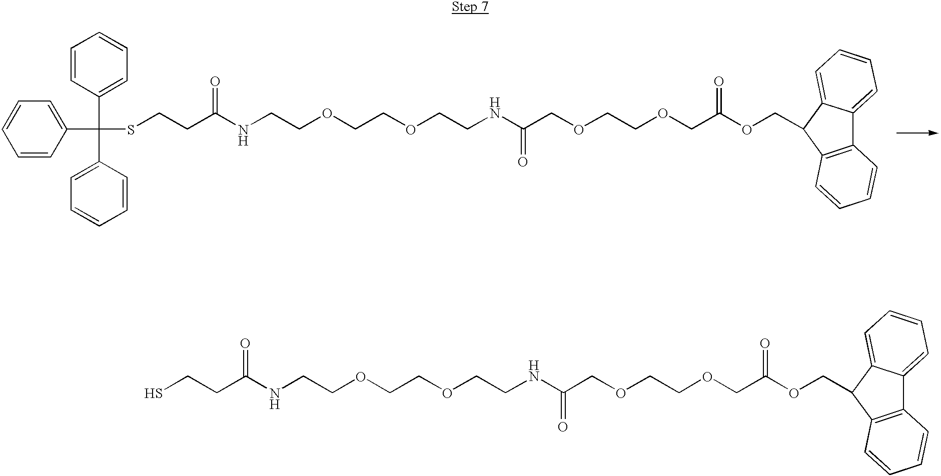

10 ml trifluoroacetic acid was added dropwise to a solution of 6.9 g product of step 6 (8.4 mmol) and 3.5 ml triethylsilane (21.4 mmol) in 30 ml DCM under intensive stirring. The reaction was monitored by LC/MS. After the reaction has been completed the reaction mixture was diluted with DCM and washed two times with 1M hydrochloric acid. The organic phase was dried with sodium sulphate over night. Then the solvent was evaporated. The residue was purified by silica column chromatography using 250 g silica and successively the following eluents: a) 2.0 l ethyl acetate; b) 1.5 l ethyl acetate/methanol (90:10); 2.0 l ethyl acetate/methanol (80:20). The yield of the purified product was 2.8 g (57.7%).

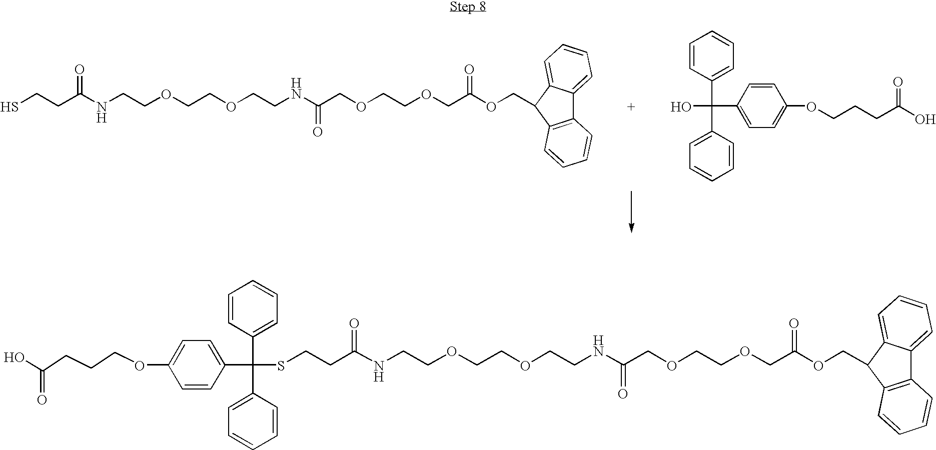

2,65 g 3-(4-(diphenylhydroxymethyl-phenoxy-)butyric acid dicyclohexylamine salt (5 mmol) was distributed in a mixture of 1M hydrochloric acid and ethyl acetate. The aqueous phase was washed twice with ethyl acetate. The combined ethyl acetate phases were dried with sodium sulphate. Then the solvent was removed under reduced pressure and the residue was dissolved in DCM. Then 2.8 g product of step 7 (5 mmol) was added to the solution and the mixture was stirred for additional 10 minutes. Then 5 ml trifluoroacetic acid was added dropwise to the reaction mixture, which became red during the reaction. The reaction was monitored by LC/MS. When the reaction has finished, the reaction mixture was diluted with DCM and washed twice with 1M hydrochloric acid. The organic phase was dried with sodium sulphate and the solvent removed under reduced pressure. The product was purified by silica column chromatography using 300 g silica and subsequently the following eluents: a) 2.0 l ethyl acetate; b) 1.5 l ethyl acetate/methanol (90:10); 1.0 l ethyl acetate/methanol (85:15); c) 2.0 l ethyl acetate/methanol (80:20). After purification 2.5 g of a pure fraction (yield=53.9%) and 1.3 g of a fraction containing impurities (28.0%) could be isolated.

Synthesis of a ligand-tag/linker conjugate X-Z-A-Y-Fmoc where Y is an amino group protected with the Fmoc-protecting group:

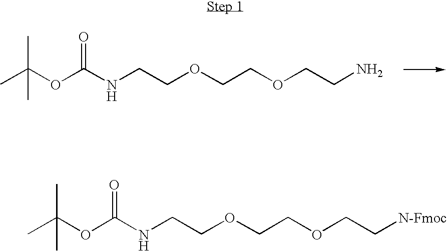

A solution of 1.68 g Fmoc-N-Hydroxysuccinimide (5.0 mmol) in 30 ml DCM was added dropwise to the intense stirred solution of 1.30 g product from step 2 (5.2 mmol) of the synthesis of the ligand-tag linker conjungate X-Z-A-Y-Fm and 0.9 ml DIEA (diisopropylethylamine) (5.2 mmol). The reaction mixture stirred over night, then the solvent was removed under reduced pressure. The residue was dissolved in ethyl acetate and washed two times with 1 M hydrochloric acid. The organic phase was dried with sodium sulphate over night. Then the solvent was evaporated under reduced pressure. The resulting raw product (2.27 g; yield=92%) was processed in the next step without further purification.

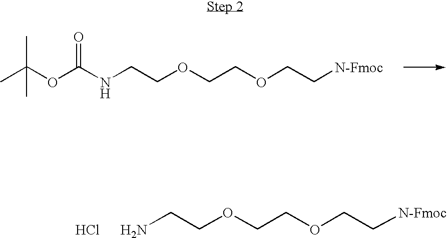

4M hydrochloric acid in dioxane was added to the solution of 2.27 g product from step 1 (4.8 mmol) in DCM and the reaction was monitored by thin layer chromatography. After the end of the reaction the solvents were removed under reduced pressure resulting in 1.8 g of a white amorphous solid (yield=92%).

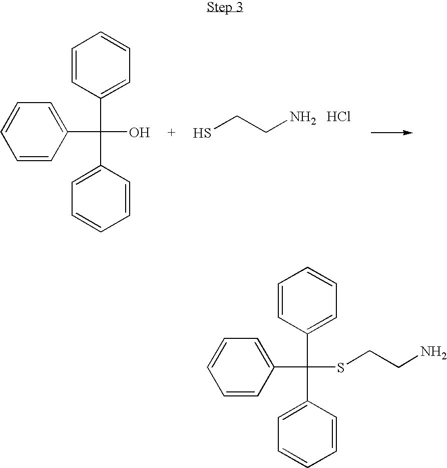

23 ml trifluoroacetic acid was added dropwise to the dispersion of 20.0 g triphenylmethanol (77 mmol) and 13.1 g cysteamine hydrochloride (115 mmol) in 350 ml DCM under intense stirring. After the addition the reaction mixture, which turned to a yellow clear solution, was stirred for additional two hours. Then the solvent was removed under reduced pressure. The product was dissolved in ethyl acetate and washed three times with a 10% solution of sodium carbonate in water. The organic phase was dried with sodium sulphate over night. The solvent was evaporated and the raw product (23.0 g; yield=93%/o) was crystallised from ethyl acetate.

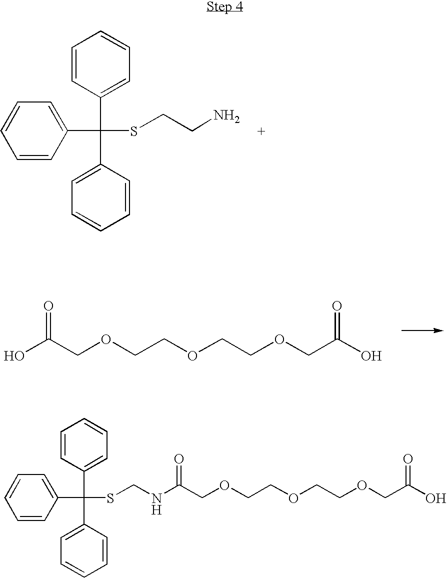

The solution of 10.2 g carbonyldiimidazole (63 mmol) in 150 ml THF was added slowly to the solution of 38.0 g 3,6,9-trioxaundecanedicarboxylic acid (171 mmol) in 250 ml THF. Then the mixture was stirred for 1 hour before (the mixture of) 49 ml DIEA (diisopropylethylamine) (285 mmol) in 60 ml THF was added. Then 18.2 g product of step 3 (57 mmol) dissolved in 300 ml THF was added slowly. The reaction mixture stirred overnight before the solvent was evaporated under reduced pressure. The product was dissolved in ethyl acetate and washed three times with 1 M hydrochloric acid. The organic phase was dried with sodium sulphate over night. The solvent was evaporated under reduced pressure. The product was purified on a silica chromatography column using 300 g silica and subsequently the following eluents: a) 1.5 l trichlormethane; b) 1.5 l trichlormethane/methanol (90:10); 1.0 l trichlormethane/methanol (80:20); c) 1.5 l trichlormethane/methanol (50:50) and d) 1.5 l methanol/1% formic acid. After purification the product yield was 14.0 g of a white powder (=47.0%).

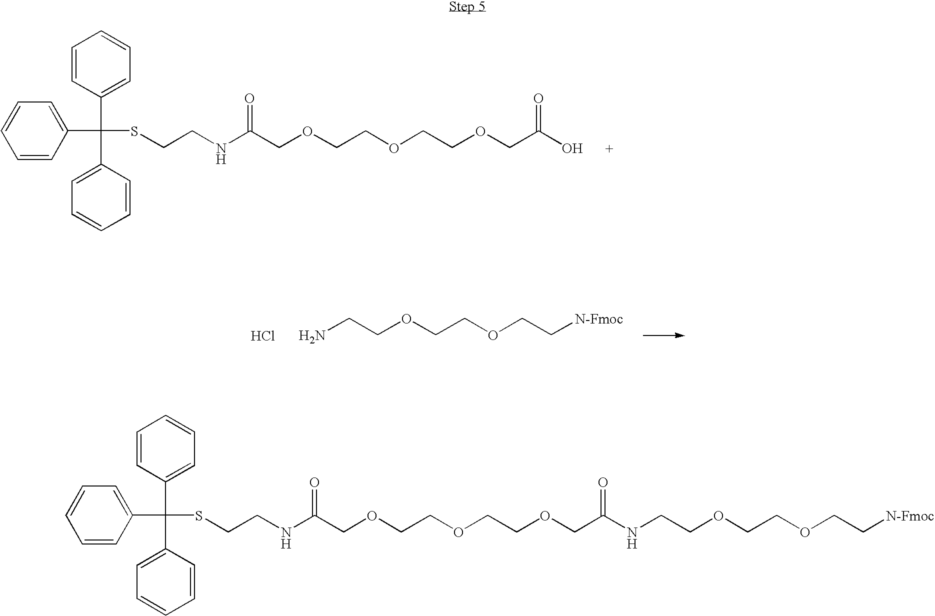

3,45 g carbonyldiimidazole (21.0 mmol) was added in small amounts to 10.0 g of product of step 4 (19.1 mmol) dissolved in 150 ml THF. Then 7.75 g of product of step 2 (19.1 mmol) dissolved in 3.3 ml DIEA (19.1 mmol) and THF was added to the mixture. After stirring over night the solvent was evaporated under reduced pressure. The residue was dissolved in ethyl acetate and washed twice with 1M hydrochloric acid in aqueous saturated sodium chloride and twice with 10% sodium carbonate in aqueous saturated sodium chloride. The organic phase was dried with sodium sulphate over night. The solvent was evaporated under reduced pressure. The raw product (14.2 g; yield=85%) was used in the next step without further purification.

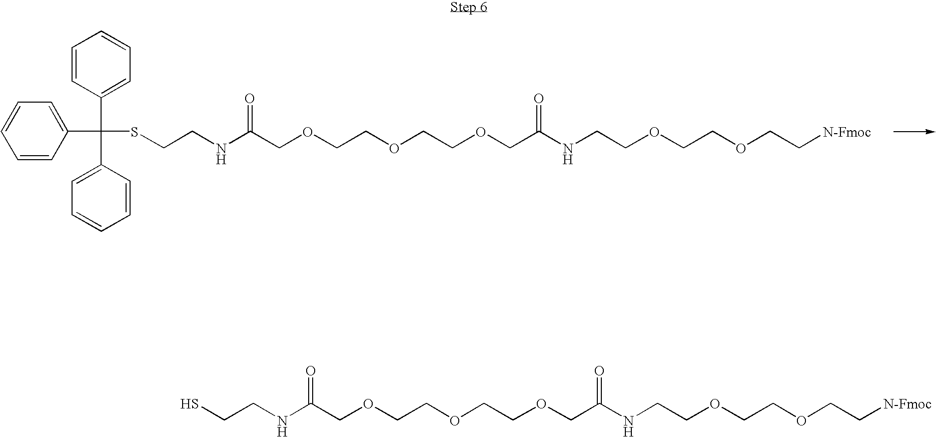

10 ml trifluoroacetic acid was added dropwise to the intense stirred solution of 14.2 g of product from step 5 (16.2 mmol) and 8.5 ml triethylsilane (50.3 mmol) in 30 ml DCM. The reaction was monitored by LC/MS. After the reaction has been completed the reaction mixture was diluted with DCM and washed two times with 1M hydrochloric acid. The organic phase was dried with sodium sulphate over night. The solvent was evaporated. The raw product was purified by silica column chromatography using 250 g silica and successively the following eluents: a) 1.5 l ethyl acetate; b) 1.5 l ethyl acetate/methanol (90:10); 2.0 l ethyl acetate/methanol (80:20).

The yield of the purified product was 8.4 g (82%).

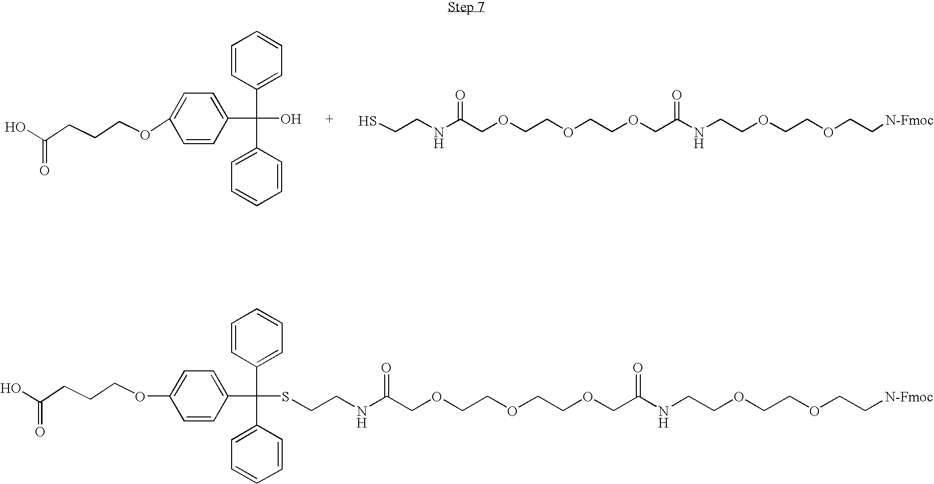

5.76 g 3-(4-(diphenylhydroxymethyl-phenoxy-)butyric acid dicyclohexylamine salt (10.6 mmol) was distributed between a mixture of 1M hydrochloric acid and ethyl acetate. The aqueous phase was washed twice with ethyl acetate. The combined ethyl acetate phases were dried with sodium sulphate. Then the solvent was removed under reduced pressure and the residue dissolved in DCM. Then 8.4 g product of step 6 (13.3 mmol) was added to the solution and the mixture was stirred for additional 10 minutes. Then 6 ml trifluoroacetic acid was added dropwise to the reaction mixture, which became red during the reaction. The reaction was monitored by LC/MS. When the reaction has finished, the reaction mixture was diluted with DCM and washed twice with 1M hydrochloric acid. The organic phase was dried with sodium sulphate and the solvent removed under reduced pressure. The product was purified by silica column chromatography using 300 g silica and subsequently the following eluents: a) 1.0 l ethyl acetate; b) 1.0 l ethyl acetate/methanol (975:25); c) 1.0 l ethyl acetate/methanol (950:50); d) 1.0 l ethyl acetate/methanol (90:10) and e) 1.0 l ethyl acetate/methanol (80:20).

After purification 5.0 g of a pure fraction (yield=48%) and 4.0 g of a impure fraction (38.0%) could be isolated.

### Solid Phase Synthesis

### 9216-Compound Library

A set of 24 384-multiwell microtiter plates (Greiner) made of polypropylene with immobilised polypropylene membrane spots (3 mm²), functionalised with amino-groups as described in DE 101 08 892.2 were used. The membranes were washed twice with DMF (dimethylformamide), then twice with DCM. 2 μl of a solution containing 55 mmol Fmoc-protected “Ligand Tag”, 55 mmol HATU (O-(7-azabenzotriazol-1-yl)-1.1.3.3-tetramethyluronium-hexafluorophospate), 11 mmol DIEA in DMF was transferred via a pipetting robot (Cybio AG, Germany). The reaction time was 1 hour. The membranes were washed six times with DMF, two times with DCM and were then air-dried.

The removal of the Fmoc protecting groups was done-with 15 μl of 25% piperidine in DMF. The cleavage reaction was performed for 20 min, then the excess piperidine was removed. The membranes were washed six times with DMF, two times with DCM and then air-dried.

For coupling of a set of 96 Fmoc-protected amino acids, 3 μl of a solution containing 0.15 M of the respective amino acid, 0.15 M HOBT and 0.16 M DIC (diisopropylcarbodiimid) in DMF was pipetted onto the membrane. After an incubation time of 1 h, the membranes were washed 6× with DW, twice with DCM and then air-dried. The Fmoc protecting groups were removed as described above. The membranes were washed six times with DMW, two times with DCM and then air-dried.

For the next coupling, a set of 88 carboxylic acids and 8 sulfonyl chlorides was used. The carboxylic acids were reacted as a 0.15 M solution in DMF, containing 0.15 M HOBT and 0.16 MDIC.

The sulfonyl chlorides were reacted as a 0.125 M solution in DMF, containing a 1.1 M surplus of DIEA N-ethyl-diisopropylamine). The carboxylic acids were coupled for 1 h, the sulfonyl chlorides for ½ h. The membranes were then washed six times with DMF, two times with DCM and then air-dried.

### Cleavage of the Ligand-Tag Conjugates from the Membrane and Removal of the Protecting Groups

A solution containing 80% TFA, 10% DCE (Dichlorethane), 5% Et₃SiH and 5% H₂O was pipetted onto the membranes. After a 1 h incubation period, the solution was removed under vacuum. For storage and further usage, the ligand-tag conjugates were dissolved in a mixture of 70% ACN and 30% H₂O containing 0.1% TFA. The ligand bearing ligand-tags have the following general formula 1:

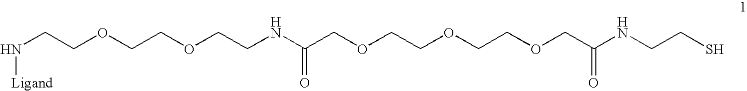

### Maleimide-Thiol Coated Plates

A gold chip (5×5 cm) was incubated with a 1:25 mixture of maleimide-thiol anchor molecules 2 and a dilution compound 3 in ethylenglycol and 1% TFA (total concentration 1.0 mM). The anchor molecule and the dilution compound were synthesised as described in examples 1 and 2 of DE 100 27 397.1. The chip was washed several times in a methanol/1% TFA mixture and then washed once in H₂O (pH 7.0). The chip was then dried under nitrogen.

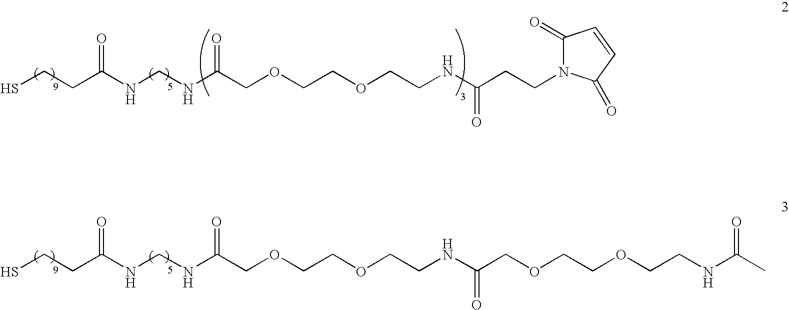

The library of 9216 ligand-tag conjugates (ligand bearing ligand-tag) with the molecular weight distribution shown in FIG. 4 was spotted on such a chip via a pin tool, thus forming an array of 96×96 spots with a spot distance of 0.575 mm. The ligand-tag conjugates were diluted to a final concentration of 40 μM in 0.2 M phosphate buffer (pi), 5 mM EDTA and 10% (v/v) ethylenglycol pH 7.0. The spot volume is approximately 10 nl, so that each spot contains a surplus of the ligand-tag conjugate compared to the surface-bound maleimide group. Thereby, a complete reaction of the maleimide groups can be obtained. In the non-occupied spaces the maleimide groups were saturated by incubating the chip in 0.2 M Pi (pH 7.0), 10 mM mercaptoethanol for 30 min.

The Chip was then treated overnight in Bovine-Serum-Albumin (BSA)-containing blocking solution (50 mM Tris/HCl, 150 mM NaCl, 5 g/l BSA, 0.05% (v/v) Tween-20, pH 7, 3). The analysis of potential binding partners of the target protein thrombin occurred by an immunoassay: Therefore, the chip was incubated for 4 h in 10 nM thrombin in blocking solution. After washing for 2 min in blocking solution, the chip was incubated with a polyclonal anti-thrombin antibody (dilution 1:1000) for 2 h. After washing two times in blocking solution the chip was incubated with an anti-rabbit-antibody-POD conjugate. Finally, the chip was washed 2× in TBST (Tris Buffered Saline with Tween) and the chemiluminescence reaction was detected_via a Lumi Imager (Roche). Bright spots show thrombin binding. A second chip was treated identically except for the incubation with thrombin. This chip served as negative control in order to differentiate binding interactions that did not occur because of thrombin binding but because of binding of the primary or secondary antibody. The negative control did not show signals above the noise level. As each compound on the array corresponds to a distinct spatial co-ordinate, the spots can be assigned to a certain chemical structure.

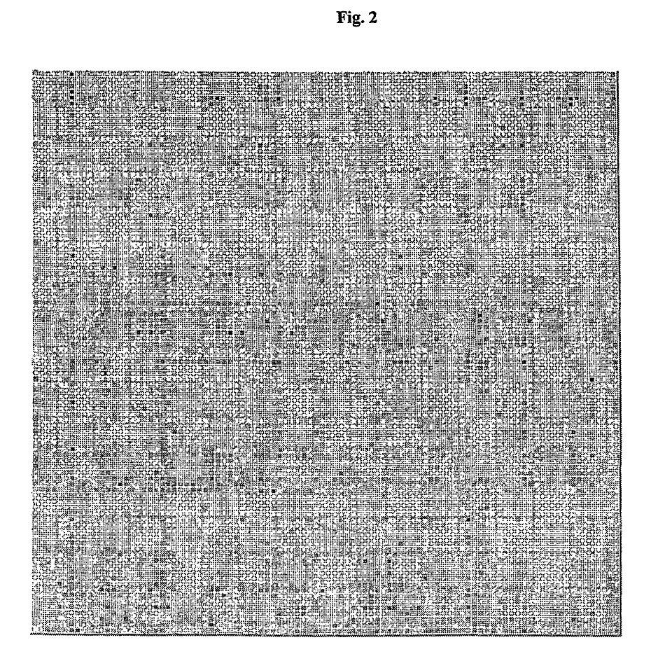

FIG. 2 shows a Jarray plot of the chemiluminescence reaction of the positive control (10 nM thrombin). Discrete intensities can be recognised at certain positions. This reveals that the substance immobilised on this position binds to thrombin. The most intensive spots were coloured in black.

### Determination of the Inhibitory Constant

The inhibitory properties of the substances identified in the direct binding assay were subsequently analysed by a thrombin assay (determination of the inhibitory constant Kᵢ). Therefor, the corresponding substances were released from the synthetic solid phase resulting in an amid group which is in common for all chemical structures. The thrombin activity is determined at 20° C. and pH 7.4 with the fluorogenic substrate Tos-GPR-AMC (Bachem, I 1365, λexc.=370 nm, λem.=450 nm). The reaction was carried out with 20 μM substrate, 0.1-100 μM Inhibitor and 100 μM human thrombin in a total volume of 200 μl BBS (10 mM Hepes, 150 mM NaCl, 0.005% Tween 20).

The reaction is started after a five-minute preincubation period of the enzyme with the inhibitor by the addition of substrate. The fluorescence intensity is measured in one-minute-intervals for 10 minutes. The Kᵢ value for competitive inhibition is calculated in the following way:

v₀/vᵢ=1+I/Ki

v₀ initial velocity of the reaction

vᵢ=initial velocity of the reaction in the presence of inhibitor

I=inhibitor concentration

The following low affinity binding ligands were identified:

| Ki (M) | compound Nr |
|---|---|
| E-03 | L1 |
| E-04 | L2 |
| 5 E-04 | L3 |
| 4 E-04 | L4 |
| 8 E-04 | L5 |
| E-03 | L6 |
| E-04 | L7 |
| E-03 | L8 |
| E-03 | L9 |
| E-03 | L10 |
| 7 E-04 | L11 |
| E-03 | L12 |
| 2 E-04 | L13 |
| E-03 | L14 |
| 5 E-04 | L15 |
| 2 E-04 | L16 |
| 7 E-04 | L17 |
| 8 E-04 | L18 |
| 3 E-04 | L19 |

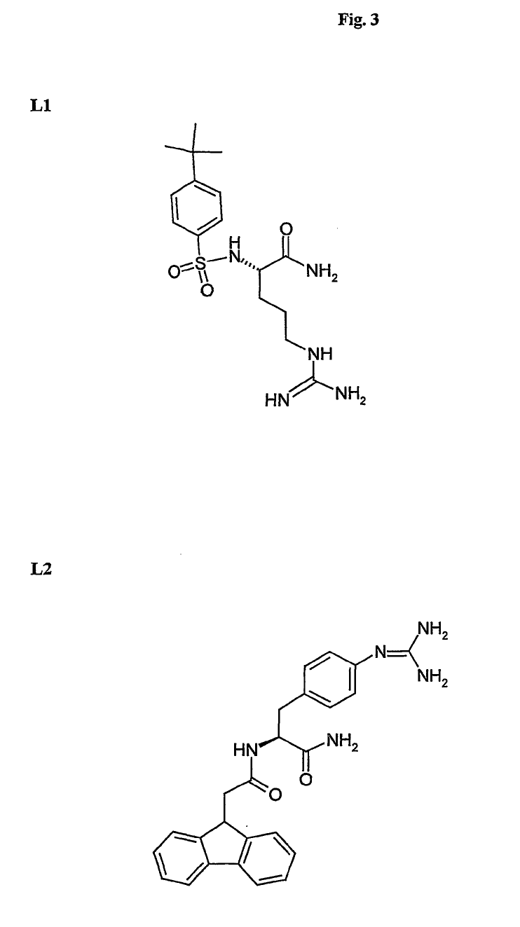

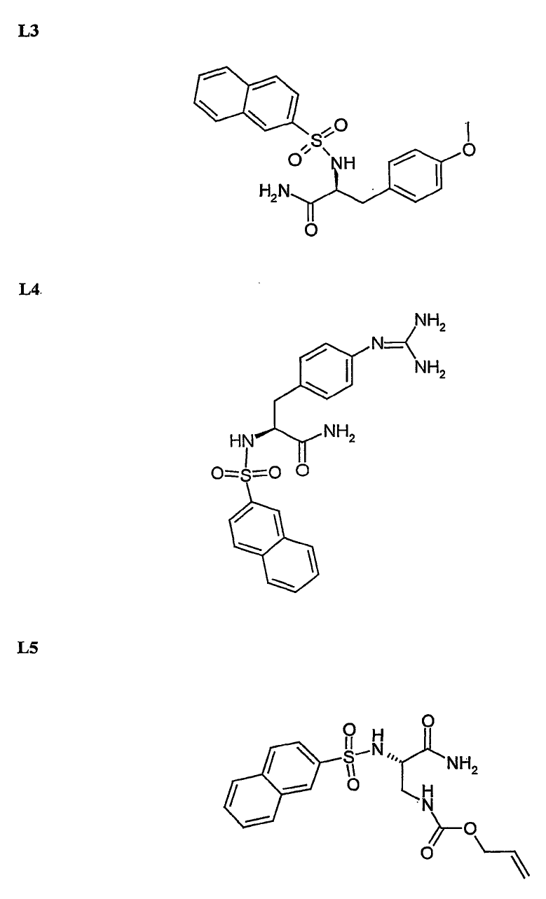

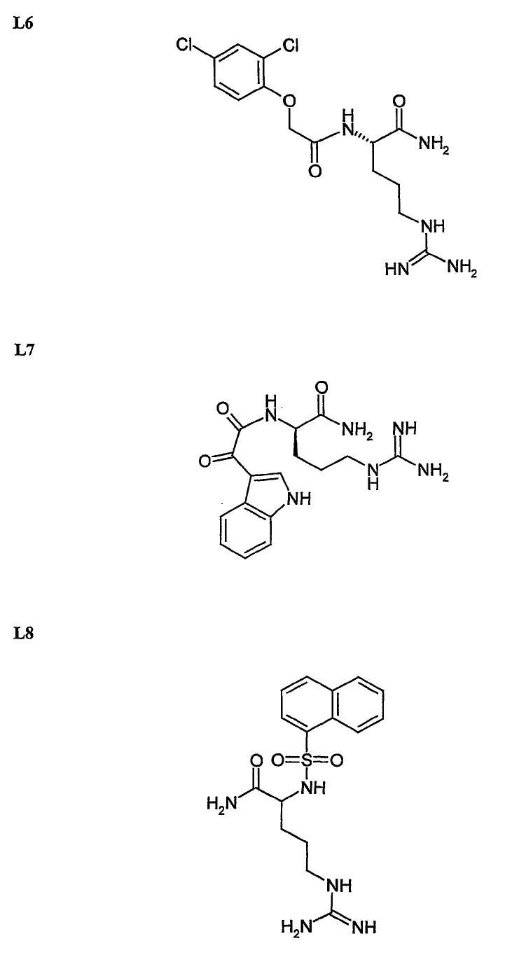

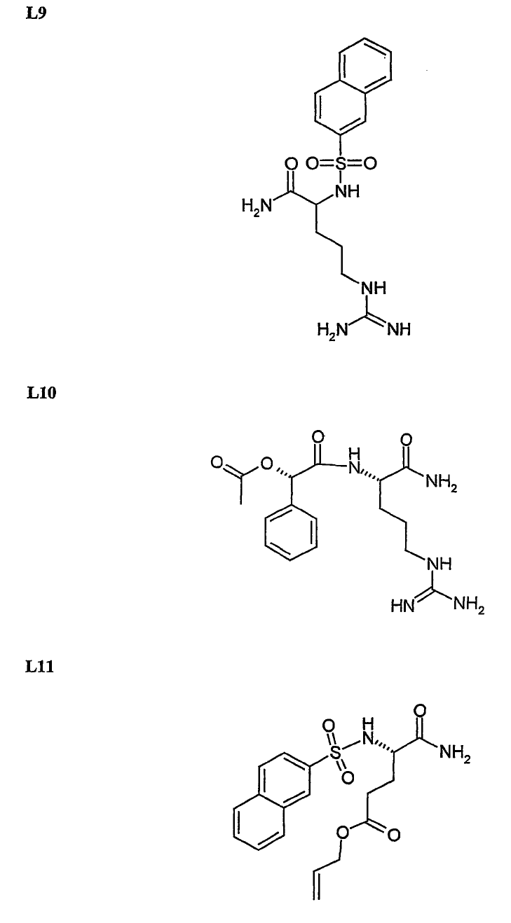

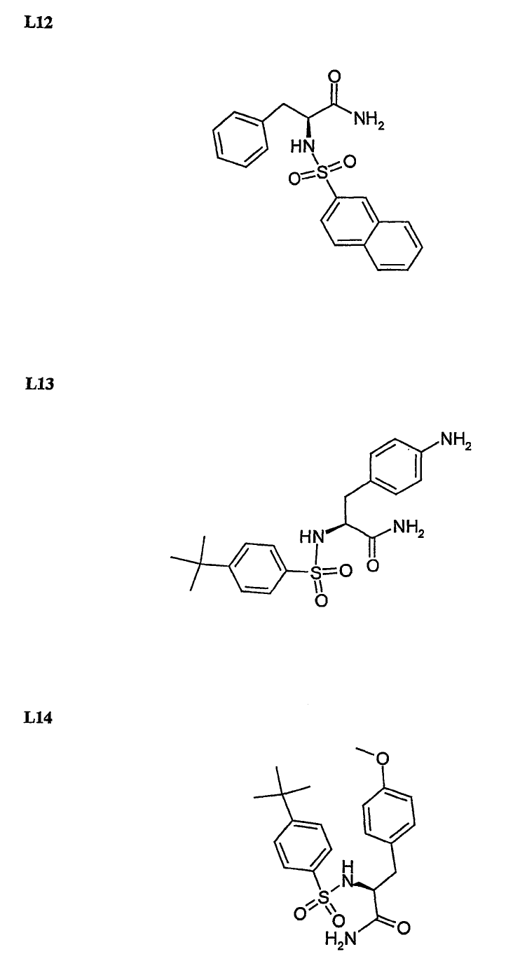

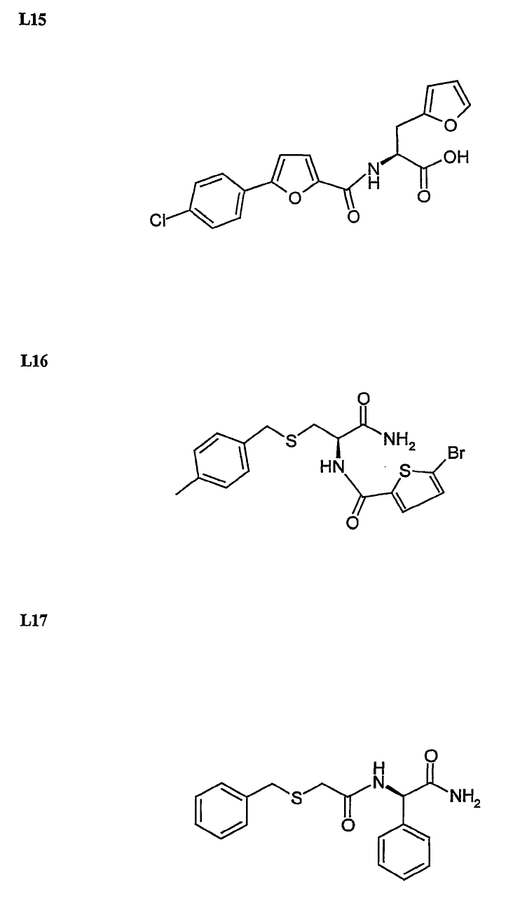

The corresponding structures are listed in FIG. 3.

1) A screening method for the detection of low affinity binding ligands, comprising the steps of:
a) providing a library of different ligands;
b) forming a binding matrix comprising the ligands on a solid support by immobilising said ligands on the support;
c) contacting a target of interest with said binding matrix;
d) parallely determining a binding value of the ligand/target interaction for each type of ligand comprised in the binding matrix;
e) selecting those ligands the binding value of which in an immobilised state towards the target exceeds a predetermined threshold;
f) evaluating the affinity of each of the ligands selected in step (e) in a non-immobilised state towards the target;
g) identifying at least one ligand of step (f) as low affinity binding ligand.

2) The method of claim 1, wherein the ligands of step (a) have a number-average molecular weight of <400 g/mol.

3) The method of claim 1 or 2, wherein the binding matrix of step (b) comprises ligands immobilized on the solid support via anchor structures which allow the formation of a self assembling monolayer on the support.

4) The method of claim 3, wherein the binding matrix is provided via immobilisation of anchor structures presenting an activated head group, followed by covalent coupling of the ligands with the anchors via reaction with these head groups.

5) The method of claim 4, wherein the ligands are coupled with the anchors by means of a ligand tag of the following structure
Z-A-Y, (6)
wherein
A is a chemical bond or a hydrocarbon chain of 2 to 50, preferably 5 to 30 C-atoms, optionally interrupted by heteroatoms, amide or ester bonds,
Y is a functional group to react with the ligand, and
Z is a functional group which is able to react with the head group of a corresponding anchor molecule.

6) The method of any of claims 1 to 5, wherein the binding matrix of step (b) further comprises dilution compounds.

7) The method according to any of claims 1 to 6, wherein the ligands of the library of step a) are synthesised via binary combinatorial synthesis starting from two sets of reactants.

8) The method according to claim 7, wherein the selection of step e) is supported by a system carrying out a step e1) of processing and visualising the binding values obtained in step d).

9) The method of claim 8, wherein the binding values are visualised in a x,y-table.

10) The method according to claim 9, wherein the x-coordinates represent a first set of reagents used in the binary combinatorial synthesis of the ligands immobilised in step b) and the y-coordinates represent a second set of reagents used in the binary combinatorial synthesis of the ligands immobilised in step b).

11) The method according to any of claims 9 or 10, wherein each cell of the x,y-table represents one of the ligands immobilised in step b) and the binding value of the ligand towards the target is visualised in a colour resolved manner.

12) A computer program comprising program code means for performing step e1) of any of claims 8 to 11 when said program is run on a computer.

13) A computer program product comprising program code means stored on a computer readable medium for performing step e1) of any of claims 8 to 11 when said program product is run on a computer.

14) A ligand-tag of the following structure:
Z-A-Y, (6)
wherein
Z is a thiol, carboxyl or amino group,
A is a chemical bond or a hydrocarbon chain of 2 to 50, preferably 5 to 30 C-atoms, optionally comprising one or more heteroatoms, amide or ester bonds, and
Y is a primary or secondary amino, carboxylic acid, hydroxyl, hydroxylamino, ester or aldehyde group.

15) A ligand/ligand-tag conjugate obtainable by covalently binding a ligand to be tested in a solid phase screening method to the group Y of the ligand-tag of claim 14.

16) A ligand/ligand-tag conjugate according to claim 15 having the structure
Z-A-Y′-L, (8)
wherein Z and A are defined as in formula (6) of claim 14, Y′ is a amide or ester bond obtainable from the reaction of group Y of formula (6) of claim 14 with a corresponding functional group of the ligand and L is a ligand structure obtainable by reacting an alcohol, a primary or secondary amine, a carboxylic acid, a carboxylic acid ester, an aldehyde or another carbonyl compound with a ligand tag of formula (6) of claim 14.

17) A ligand/ligand-tag conjugate according to claim 16, having the following structure
Z-A-HNC(O)-L²-L¹, (9)
wherein
Z is a thiol, carboxyl or amino group,
A is a chemical bond or a hydrocarbon chain of 2 to 50, preferably 5 to 30 C-atoms, optionally comprising one or more heteroatoms, amide or ester bonds, and
L² is an amino acid residue using its amino functional group to form an amide or sulfonamide bond with L¹ and its carboxylic functional group to form an amide bond with the remaining structure Z-A-HN and
L¹ is a carboxylic or sulfonic acid compound using its functional group to complete the amide or sulfonamide bond.

18) A library for use in a solid phase screening method formed by a plurality of different ligand/ligand-tag conjugates of any of claims 15 to 17.

19) An array for use in a solid phase screening method, comprising a plurality of members of the library of claim 18 immobilised on a solid support

20) A screening chip, comprising an array according to claim 19.

21) A Method for providing a binding matrix to be used in a screening process on a solid support, comprising the steps of
a) covalently coupling the ligands to be screened to the functional group Y of the ligand tag of claim 14 to form ligand/ligand-tag conjugates,
b) immobilizing anchor structures on the support which allow the formation of a self assembling monolayer and which present an activated head group capable of reacting with the functional group Z of the ligand tag of claim 14.
c) covalently coupling the ligand/ligand-tag conjugates with the anchors immobilized on the support.
